SEQUENCE LISTING (5 sequences)

SEQ ID NO: 1 - RNA, 30 nt, synthetic construct
  modified_base 30: mod_base = OTHER; note = thymine
  misc_feature 1..30: note = Description of Artificial Sequence: Syntheticoligonucleotide
  misc_feature 1..30: note = Description of Combined DNA/RNA Molecule: Syntheticoligonucleotide
  modified_base 1..29: mod_base = OTHER; note = 2'-O-Methyl nucleotide
  modified_base 30: mod_base = OTHER; note = Inverted nucleotide
  source 1..30: mol_type = other RNA
gccagggacctaagacacatgtccctggct

SEQ ID NO: 2 - RNA, 30 nt, synthetic construct; identical to SEQ ID NO: 1
  modified_base 30: mod_base = OTHER; note = thymine
  misc_feature 1..30: note = Description of Artificial Sequence: Syntheticoligonucleotide
  misc_feature 1..30: note = Description of Combined DNA/RNA Molecule: Syntheticoligonucleotide
  modified_base 1..29: mod_base = OTHER; note = 2'-O-Methyl nucleotide
  modified_base 30: mod_base = OTHER; note = Inverted nucleotide
  source 1..30: mol_type = other RNA

SEQ ID NO: 3 - RNA, 30 nt, synthetic construct; identical to SEQ ID NO: 1
  modified_base 30: mod_base = OTHER; note = thymine
  misc_feature 1..30: note = Description of Artificial Sequence: Syntheticoligonucleotide
  misc_feature 1..30: note = Description of Combined DNA/RNA Molecule: Syntheticoligonucleotide
  modified_base 1..29: mod_base = OTHER; note = 2'-O-Methyl nucleotide
  modified_base 30: mod_base = OTHER; note = Inverted nucleotide
  source 1..30: mol_type = other RNA

SEQ ID NO: 4 - RNA, 30 nt, synthetic construct; identical to SEQ ID NO: 1
  modified_base 30: mod_base = OTHER; note = thymine
  misc_feature 1..30: note = Description of Artificial Sequence: Syntheticoligonucleotide
  misc_feature 1..30: note = Description of Combined DNA/RNA Molecule: Syntheticoligonucleotide
  modified_base 1..29: mod_base = OTHER; note = 2'-O-Methyl nucleotide
  modified_base 30: mod_base = OTHER; note = Inverted nucleotide
  source 1..30: mol_type = other RNA

SEQ ID NO: 5 - RNA, 30 nt, synthetic construct; identical to SEQ ID NO: 1
  misc_feature 1..30: note = Description of Artificial Sequence: Syntheticoligonucleotide
  misc_feature 1..30: note = Description of Combined DNA/RNA Molecule: Syntheticoligonucleotide
  modified_base 1..29: mod_base = OTHER; note = 2'-O-Methyl nucleotide
  modified_base 30: mod_base = OTHER; note = Inverted nucleotide
  source 1..30: mol_type = other RNA
  modified_base 30: mod_base = OTHER; note = thymine

(19) United States
(12) Patent Application Publication
(10) Pub. No.: US 2024/0247274 A1
(43) Pub. Date: Jul. 25, 2024

(54) FATTY ACID CONJUGATES OF NUCLEIC ACIDS

(71) Applicant: Guardian Therapeutics, LLC, Lexington, MA (US)

(72) Inventor: Shuhao ZHU, Concord, MA (US)

(73) Assignee: Guardian Therapeutics, LLC, Lexington, MA (US)

(21) Appl. No.: 18/599,808

(22) Filed: Mar. 8, 2024

Related U.S. Application Data
(63) Continuation of application No. PCT/US2022/076198, filed on Sep. 9, 2022.
(60) Provisional application No. 63/242,679, filed on Sep. 10, 2021.

(51) Int. Cl. C12N 15/115; C12N 15/113

(52) U.S. Cl. CPC C12N 15/115; C12N 15/113; C12N 2310/14; C12N 2310/141; C12N 2310/3515; C12N 2310/531; C12N 2310/532

(57) ABSTRACT

The present disclosure relates to a conjugate comprising a fatty acid moiety and a nucleic acid moiety wherein the fatty acid moiety is conjugated to the nucleic acid via a polyethylene glycol (PEG) and a glutamic acid group. The nucleic acid moiety can be an aptamer or a variant thereof, an oligonucleotide, an antisense oligonucleotide, a CpG oligonucleotide, and a therapeutic RNA such as mRNA, siRNA, shRNA, microRNA, lncRNA, saRNA, circular RNA and the like. Methods for manufacturing the conjugates and use thereof are also provided.

### CROSS REFERENCE TO RELATED APPLICATIONS

This application is a continuation application of International Application No. PCT/US2022/076198, filed Sep. 9, 2022, which claims priority to U.S. Provisional Application No. 63/242,679, filed on Sep. 10, 2021, the contents of each of which are incorporated herein by reference in their entireties.

### REFERENCE TO THE SEQUENCE LISTING

The present application is being filed along with a Sequence Listing which has been submitted electronically in XML format. Said XML copy, created on Mar. 8, 2024, is named GBT-003US1.XML and is 17,938 bytes in size; the contents of which are hereby incorporated by reference in their entirety.

### BACKGROUND

Nucleic acid-based therapeutics represent a novel category of drugs to target diseases and genes that are un-targetable by classic small molecule approaches. One key issue for developing nucleic acid-based drugs is to enhance its in vivo stability, half-life, clearance and tissue distribution, therefore, increasing the therapeutic efficacy of drugs.

Chemically modified nucleosides are routinely used for incorporation into nucleic acid molecules to enhance one or more properties, such as nuclease resistance, pharmacokinetics or affinity to a target. In some cases, additional modifications, such as chemical moieties, are conjugated to nucleic acid therapeutics to improve the potency and efficacy of nucleic acid compounds.

The long blood circulatory property of human serum albumin provides an attractive drug half-life extension enabling technology. One strategy to increase the albumin binding is to modify a target drug with fatty acid conjugates, which can bind to albumin. A successful example is Semaglutide, a glucagon-like peptide-1 receptor agonist (GLP-1 RA). Human GLP-1 is rapidly degraded by dipeptidyl peptidase-4 (DPP-4) enzyme, resulting in a short half-life. Semaglutide, the human GLP-1 analog with 94% homolog, includes two structural modifications: replacement of Gly with the non-proteinogenic amino acid 2-aminoisobutyric acid (Aib) at position 2 and the attachment of octadecanoic diacid to the side chain of Lys-26 through a short polyethylene glycol (PEG) spacer and a γ-glutamic acid linker. The presence of the 18-carbon fatty acid moiety results in a high binding affinity for serum albumin, which translates to a half-life of approximately 7 days in humans (Witteloostuijn et al., Half-life extension of biopharmaceuticals using chemical methods: Alternatives to PEGylation. Chem Med Chem., 2016, 11(22):2474-2495).

Similarly, lipid conjugations including fatty acid conjugations have been shown to impact therapeutic nucleic acids like siNRAs (e.g., Biscans et al., The valency of fatty acid conjugates impact siRNA pharmacokinetics, distribution, and efficacy in vivo. J. Control Release, 2019, 302:116-125).

The present disclosure relates to novel fatty acid conjugations to therapeutic nucleic acids, e.g., aptamers and small RNA molecules. Such fatty acid moieties impact in vivo efficacy such as the extension of the half-life of modified aptamers in the blood.

### BRIEF DESCRIPTION OF THE DRAWINGS

FIG. 1 shows fatty acid conjugates BT500, BT600 and BT700 binding to albumin. BT100 is tested as control.

### SUMMARY OF THE INVENTION

The present invention provides novel fatty acids that can be used to create nucleic acid-fatty acid conjugates for increasing the performance of nucleic acid therapeutics, for example, extending the half-life in vivo.

The present invention provides conjugates comprising a nucleic acid moiety and at least one fatty acid moiety in which the fatty acid moiety is covalently conjugated to the nucleic acid moiety, and compositions and methods of use of the same. The fatty acid moieties modify the features of the nucleic acid moiety of the conjugate e.g., half-life.

In some embodiments, the fatty acid moiety comprises —(CH₂)ₐ—COOH, wherein a is an integer between 12 and 26. The conjugate may comprise at least one additional carboxyl group. In some examples, the conjugate may comprise one or two additional carboxyl groups. In one embodiment, the conjugate comprises one additional carboxyl group. In another embodiment, the conjugate comprises two additional carboxyl groups.

In some embodiments, the fatty acid moiety comprises at least one ethylene glycol group (—(OCH₂CH₂)—). In some examples, the fatty acid moiety comprises between 1 and 10 ethylene glycol groups, e.g., 1, 2, 3, 4, 5, 6, 7, 8, 9 or 10 ethylene glycol groups.

In some embodiments, the fatty acid moiety comprises at least one polyethylene glycol (PEG) group. The fatty acid moiety may further comprise at least one amide group (—NH—CO—).

In one embodiment, the fatty acid moiety comprises at least one —(OCH₂CH₂—OCH₂CH₂—NH—CO)— group.

In another embodiment, the fatty acid moiety comprises at least one —(CO—CH₂—OCH₂CH₂—OCH₂CH₂—NH)— group.

In yet another embodiment, the fatty acid moiety comprises at least one glutamic acid group. In some examples, the glutamic acid group is a γ-glutamic acid group.

In one embodiment, the fatty acid moiety comprises

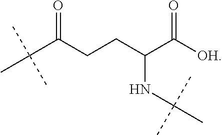

In another embodiment, the fatty acid moiety comprises

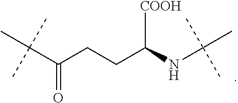

In some embodiments, the fatty acid moiety comprises a piperazine group, such as a piperazine-2-carboxylic acid group

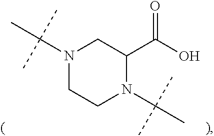

a

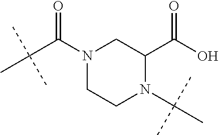

group, and a

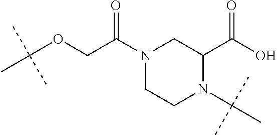

group.

In one embodiment, the conjugate comprises a fatty acid moiety comprising a structure of:

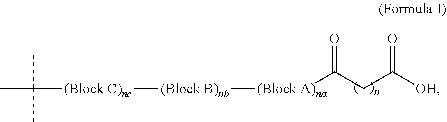

wherein n is an integer from 12 to 26,
Block A is

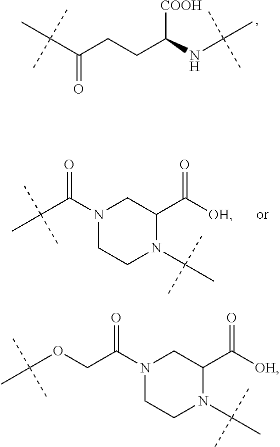

na is 0 or 1,
Block B is —(OCH₂CH₂)— or —(CO—CH₂—OCH₂CH₂—OCH₂CH₂—NH)—,
nb is an integer from 1 to 10,
Block C is

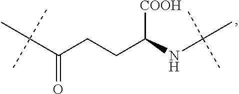

and
nc is 0 or 1.

As non-limiting examples, the conjugate comprises a fatty acid moiety selected from the group consisting of GTFA-1. GTFA-1′, GTFA-2, GTFA-2′, GTFA-3 and GTFA-3′.

The nucleic acid moiety of the present conjugate is a therapeutic nucleic acid, including but not limited to, an aptamer or a variant thereof, an oligonucleotide, an antisense oligonucleotide, a CpG oligonucleotide, a siRNA, a microRNA, a lncRNA, an mRNA, an antisense RNA, a saRNA, a circular RNA and the like.

In some embodiments, the nucleic acid moiety is an aptamer or a variant thereof. The aptamer comprises about 15-100 nucleotides, about 15-75 nucleotides, or about 15-50 nucleotides, or about 15-30 nucleotides, or about 20-50 nucleotides, or about 20-30 nucleotides.

In some embodiments, the aptamer comprises at least one chemical modification such as a nucleoside modification and a backbone modification.

In another aspects, the present invention provides compositions comprising nucleic acid-fatty acid conjugates and methods of using the compositions for therapeutics.

In some embodiments, the present invention provides a method of extending half-life of a nucleic acid molecule, the method comprising modifying the nucleic acid molecule with a fatty acid moiety.

In another aspect, the present invention provides a fatty acid moiety which comprises a general formula of:

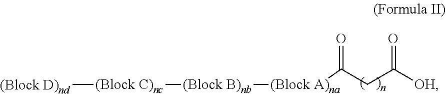

wherein n is an integer from 12 to 26,
Block A is

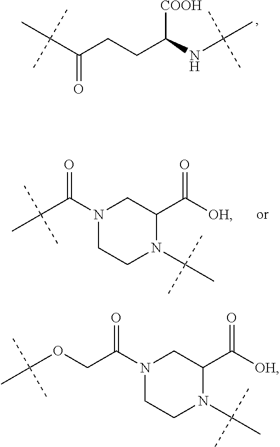

na is 0 or 1,
Block B is —(OCH₂CH₂)— or —(CO—CH₂—OCH₂CH₂—OCH₂CH₂—NH)—,
nb is an integer from 1 to 10,
Block C is

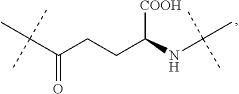

nc is 0 or 1,
Block D comprise a functional group that can covalently bind to a nucleic acid moiety, and
nd is 0 or 1.

In some embodiments, Block D of the fatty acid moiety comprises an azide group, an alkyne group, a hydroxy group, a sulfhydryl group, or an amino group. In some embodiment, the Block D of the fatty acid moiety is R_{d}—N₃, wherein R_{d} comprises an alkyl group, an amino alkyl group, an amine group, and/or an alkoxyl group. In some examples, the Block D is

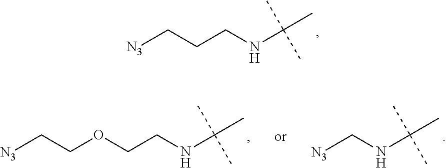

As non-limiting examples, the fatty acid moiety is GTFA-1, GTFA-1′, GTFA-2, GTFA-2′, GTFA-3, or GTFA-3′.

In another aspect, the present invention provides a conjugate comprising a polynucleotide comprising a nucleic acid sequence presented by SEQ ID NO.: 1 and a fatty acid moiety conjugated to one terminal of SEQ ID NO.:1. As non-limiting examples, the conjugates are BT500 (SEQ ID NO.: 3), BT600 (SEQ ID NO.: 4) and BT700 (SEQ ID NO.: 5). In some embodiments, a composition comprising a conjugate is provided, wherein the conjugate comprises BT500 (SEQ ID NO.: 3), BT600 (SEQ ID NO.: 4) or BT700 (SEQ ID NO.: 5).

### DETAILED DESCRIPTION OF THE DISCLOSURE

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### INTRODUCTION

Conjugation of a moiety to nucleic acid molecules such as small therapeutic nucleic acid molecules is useful to modify the features of nucleic acids, for example to increase their half-life in the body, stability, binding to a target, functional efficacy etc. Technologies to conjugate other functional molecules to nucleic acids either directly or indirectly through post-synthetic labeling and various conjugation chemistries have been greatly advanced. For example, click chemistry is among the most robust and efficient chemistries useful for biomolecular conjugations and is widely used in protein and DNA/RNA conjugations.

It was observed that free fatty acids were capable of binding with human serum albumin (HSA) (Curry S. et at, Fatty acid binding to human serum albumin: new insights from crystallographic studies. Biochim Biophys Acta, 1999, 1441: 131-140). HSA is abundant in blood and is an important player in drug metabolism (Bhattacharya, A. A., et at., Crystallographic analysis reveals common modes of binding of medium and long-chain fatty acids to human serum albumin. J Mol Biol., 2000, 303: 721-732), Most drugs are bound to HSA and are dissociated in blood circulation and for tissue transfer (Ghuman, J. et al., Structural basis of the drug-binding specificity of human serum albumin. J Mol Biol., 2005, 353:38-52.) The function of binding to HAS makes fatty acids a very useful tool to extend the blood retention time of drugs, i.e., extension of half-life. Many studies have demonstrated that conjugation of fatty acids with therapeutic agents, e.g., peptides and siRNAs, can delay the absorption rate, prolong the duration of the circulation and protect against proteolysis (e.g., Troiber, C. et at, Stabilizing effect of tyrosine trimers on pDNA and siRNA polyplexes. Biomaterials, 2013, 34: 1624-1633; and Hackett, M. J., et al., A dicarboxylic fatty acid derivative of paclitaxel for albumin-assisted drug delivery. J Pharm Sci., 2012, 101:3292-3304). It was reported that the conjugation of the C16 fatty acid resulted in a significant increase in the half-life of GLP-1 peptide (Madsen, K. et al. Structure-activity and protraction relationship of long-acting glucagon-like peptide-1 derivatives: importance of fatty acid length, polarity and bulkiness. J Med Chem, 2007, 50, doi: 10.1021/jm070861j). Fatty acid conjugation has been developed as a useful strategy to extend the half-life and the stability of therapeutic agents (e.g., peptides, DNAs and RNAs) due to this efficiency and non-toxic effects clinically.

The lengths and structures of fatty acids could impact differently the therapeutic agents. The conjugation of fatty acids with active therapeutic agents requires complex steps. In many cases, the functional groups in therapeutic agents are limited, due to its chemical and physical properties, e.g., its structural conformations. The active sites in fatty acids could also limit their applications. To overcome this obstacle, active groups can be introduced into the fatty acids and/or therapeutic agents. The modifications can be achieved through molecular modifications or a linker to conjugate agents and fatty acids.

The present disclosure provides a modified fatty acid analog and an optimized conjugating strategy for fatty acid conjugations to nucleic acid molecules, particularly small nucleic acid molecules, e.g., an aptamer, an oligonucleotide, an antisense oligonucleotide, a CpG oligonucleotide, a siRNA, a siRNA, a microRNA, a lncRNA, a mRNA, an antisense RNA, a saRNA and the like. The fatty acid-like molecules (referred to as “fatty acid moiety” herein) of the present disclosure comprise one or more functional groups such as two or more carboxyl groups, and additional active groups such as azide groups, alkyne groups, hydroxy groups, sulfhydryl groups, and amino groups.

### Definitions

To more clearly and concisely describe the subject matter of the claimed disclosure, the following definitions are provided for specific terms, which are used in the following description and the appended claims. Throughout the specification, exemplification of specific terms should be considered as non-limiting examples.

As used herein, the term “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some cases, an aliphatic group contains 2-50 aliphatic carbon atoms, or 2-20 aliphatic carbon atoms, or 8-30 aliphatic carbon atoms, or 10-20 aliphatic carbon atoms. In some embodiments, an aliphatic group contains 2, 3, 4, 5, 6, 7, 8, 9, 10, 11, 12, 13, 14, 15, 16, 17, 18, 19, 20, 21, 22, 23, 24, 25, 26, 27, 28, 29 or 30 aliphatic carbon atoms. In accordance with the present disclosure, aliphatic groups include, but are not limited to, linear or branched, substituted or unsubstituted alkyl, alkenyl, alkynyl groups and hybrids thereof.

As used herein, the term “binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁶ M¹ or lower. The term “affinity” refers to the strength of binding: increased binding affinity being correlated with a lower K_{d}.

As used herein, the term “molecular weight”, as used herein, generally refers to the mass or average mass of a material. If a polymer or oligomer, the molecular weight can refer to the relative average chain length or relative chain mass of the bulk polymer. The molecular weight of a molecule may be calculated as the sum of the atomic weight of each atom in the formula of the conjugate multiplied by the number of each atom. It may also be measured by mass spectrometry, NMR, chromatography, light scattering, viscosity, and/or any other methods known in the art. It is known in the art that the unit of molecular weight may be g/mol, Dalton (Da), or atomic mass unit (amu), wherein 1 g/mol=1 Da=1 amu.

As used herein, the term “pharmaceutical composition” refers to the combination of an active agent (e.g., a therapeutic nucleic acid) with a carrier, inert or active, making the composition especially suitable for diagnostic or therapeutic use in vivo or ex vivo. The carrier in the pharmaceutical composition must be acceptable in the sense that it is compatible with the active ingredient and capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

As used herein, the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. In some examples, the compositions and formulations also can include stabilizers and preservatives.

As used herein, the term “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans, or generally recognized as safe for use in parenteral products.

As use herein, the terms “treating”, or “treatment” refer to both therapeutic treatment and prophylactic or preventative measures. They refer to preventing, curing, reversing, attenuating, alleviating, minimizing, suppressing, or halting the deleterious effects of a disease state, disease progression, disease causative agent (e.g., bacteria or viruses), or other abnormal condition.

As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. Examples of therapeutic agents may include, but are not limited to, small molecule drugs, chemotherapeutic agents, immunotherapeutic agents, therapeutic antibodies and fragments thereof, toxins (e.g., immunotoxins), radioisotopes, enzymes (e.g., enzymes to cleave prodrugs to a cytotoxic agent at the target site), nucleases, hormones, immunomodulators, aptamers, antisense oligonucleotides, CpG oligodeoxynucleotides (or CpG ODN), nucleic acid molecules (e.g., mRNA molecules, cDNA molecules, microRNA molecules, RNAi molecules such as siRNA or shRNA, saRNA, or lncRNA molecules), chelators, boron compounds, photoactive agents and dyes. The therapeutic agent may also include a metal, metal alloy, intermetallic or core-shell nanoparticle bound to a chelator that acts as a radiosensitizer to render the targeted cells more sensitive to radiation therapy as compared to healthy cells.

As used herein, the term “therapeutically effective amount” generally refers to an amount of the aptamer of the present disclosure to affect a desired biological response. Such response may be a beneficial result, including, without limitation, amelioration, reduction, prevention, or elimination of symptoms of a disease or disorder. Therefore, the total amount of each active component of the aptamer or method is sufficient to demonstrate a meaningful benefit in a subject in need, including, but not limited to, treatment of cancer. A “therapeutically effective amount” may be administered through one or more preventative or therapeutic administrations. When a “therapeutically effective level” is applied to a single ingredient, administered alone, the term refers to that composition alone. When applied to a combination, the term refers to combined amounts of the active compositions that produce the therapeutic effect, whether administered in combination, consecutively, or simultaneously. The exact amount required will vary from subject to subject, depending, for example, on the species, age, and general condition of the subject; the severity of the condition being treated; the particular antigen of interest; in the case of an immunological response, the capacity of the subject's immune system to synthesize antibodies, for example, and the degree of protection desired; and the mode of administration, among other factors. An appropriate “effective” amount in any individual case may be determined by one of ordinary skill in the art. Thus, a “therapeutically effective amount” will typically fall in a relatively broad range that can be determined through routine trials.

As used herein, the terms “patient,” “individual,” or “subject” are used interchangeably and intended to include human and non-human animals. Exemplary human subjects include a human patient suffering from cancer, and HCC in particular. The term “non-human animals” includes all vertebrates, e.g., non-mammals (such as chickens, amphibians, reptiles) and mammals, such as non-human primates, domesticated and/or agriculturally useful animals (such as sheep, dogs, cats, rabbits, cows, pigs, etc.), and rodents (such as mice, rats, hamsters, guinea pigs, etc.

### Compositions

### Fatty Acid-Nucleic Acid Conjugates

The present disclosure relates to fatty acid-nucleic acid conjugates. A conjugate disclosed herein comprises at least one fatty acid moiety and a nucleic acid moiety wherein the fatty acid moiety is conjugated to the nucleic acid. Preferably, the fatty acid moiety is conjugated to one end of the nucleic acid. The conjugate has an extended half-life compared with the nucleic acid alone without the fatty acid moiety. Compositions comprising fatty acid-nucleic acid conjugates as described herein are provided as well.

As used herein, the term “conjugate” is used to refer to two or more entities (e.g., moieties) that are linked by direct or indirect covalent or non-covalent interaction. In some embodiments, the interaction is covalent. In some embodiments, a covalent interaction is mediated by a linker moiety. In some embodiments, the interaction is non-covalent (e.g., charge interactions, affinity interactions, metal coordination, physical adsorption, host-guest interactions, hydrophobic interactions, stacking interactions, hydrogen bonding interactions such as with “sticky sequences,” van der Waals interactions, magnetic interactions, electrostatic interactions, dipole-dipole interactions, etc.).

As used herein, the term “moiety” means a specific part or functional group of a molecule or a compound (e.g., a conjugate), which lacks one or more atom(s) compared to the corresponding reagent. If, for example, a reagent of the formula “H—X—H” reacts with another reagent and becomes part of the reaction product.

In accordance with the present disclosure, the terms “nucleic acid,” “oligonucleotide,” and “polynucleotide” should in no way be considered limiting and may be used interchangeably herein. A nucleic acid molecule is a polymer of nucleotides consisting of at least two nucleotides covalently linked together. A nucleic acid molecule is a DNA (deoxyribonucleotide), an RNA (ribonucleotide), as well as a recombinant RNA and DNA molecule or an analogue of DNA or RNA generated using nucleotide analogues. The nucleic acids may be single stranded or double stranded, linear or circular. The term also comprises fragments of nucleic acids, such as naturally occurring RNA or DNA which may be recovered using the extraction methods disclosed, or artificial DNA or RNA molecules that are artificially synthesized in vitro (i.e., synthetic polynucleotides). Molecular weights of nucleic acids are also not limited, may be optional in a range from several base pairs (bp) to several hundred base pairs, for example from about 2 nucleotides to about 1,0000 nucleotides, or from about 10 nucleotides to 5,000 nucleotides, or from about 10 nucleotides to about 1,000 nucleotides. “Oligonucleotide” is used when the relevant nucleic acid molecules typically comprise less than about 100 bases. “Polynucleotide” is used when the relevant nucleic acid molecules typically comprise more than about 100 bases. All the terms are used to denote DNA, RNA, modified or synthetic DNA or RNA (including, but not limited to nucleic acids comprising synthetic and naturally occurring base analogs, dideoxy or other sugars, thiols or other non-natural or natural polymer backbones), or other nucleobase containing polymers capable of hybridizing to DNA and/or RNA. Accordingly, the terms should not be construed to define or limit the length of the nucleic acids referred to and used herein, nor should the terms be used to limit the nature of the polymer backbone to which the nucleobases are attached. Types of nucleic acids include but not limited to oligonucleotides such as antisense oligonucleotides and CpG oligonucleotides, aptamers and variants thereof, small RNA molecules such as microRNAs, siRNAs, shRNAs, lncRNAs and saRNAs, mRNAs, and cDNAs.

As used herein, the term “fatty acid moiety” refers to a molecule or a radical in the context of a conjugate, wherein the molecule or radical comprises a fatty acid. In some embodiments, the fatty acid moiety comprises at least one additional carboxyl group in addition to the carboxyl group of the fatty acid. In some embodiments, the fatty acid moiety comprises one additional carboxyl group in addition to the carboxyl group of the fatty acid. In some embodiments, the fatty acid moiety comprises two additional carboxyl groups in addition to the carboxyl group of the fatty acid.

In some embodiments, the molecular weight of the conjugate is at least 5K Da, such as between 5K-10K Da, between 10K-20K Da, between 20K-30K Da, between 30K-40K Da, or between 40K-50K Da.

In some embodiments, the conjugates have at least one carboxyl group (—COOH), for example, one carboxyl group, two carboxyl groups, or three carboxyl groups.

A nucleic acid molecule, such as aptamer, may be conjugated to any suitable position of a fatty acid moiety except at the terminal —COOH, via an optional linker, to form a conjugate, as long as the function of the nucleic acid, such as the binding of the aptamer to its target, is not negatively affected. In some embodiments, the nucleic acid, e.g., an aptamer, is attached to a terminus of the fatty acid moiety. After the nucleic acid is conjugated to the fatty acid moiety, a hydrogen or a functional group at the terminus of the fatty moiety is replaced with the nucleic acid.

In some embodiments, the fatty acid moiety is conjugated to one terminal of the nucleic acid molecule. In some examples, the fatty acid moiety is conjugated to the 5′ end of the nucleic acid. In other examples, the fatty acid moiety is conjugated to the 3′ end of the nucleic acid.

In some embodiments, the fatty acid moiety is conjugated to a nucleic acid of interest via a linker. The fatty acid moiety as described herein has been found to increase the half-life of said biomolecule to a much greater extent than more commonly used fatty acid residues. In some embodiments, the fatty acid moiety binds to albumin. In some embodiments, the conjugate comprising the fatty acid moiety and the nucleic acid has low renal clearance. In some embodiments, the conjugate has lower renal clearance than the nucleic acid alone.

In some embodiments, the fatty acid moiety is covalently linked to the nucleic acid molecule described herein. The manner of binding the fatty acids to the nucleic acids is not particularly limited. The fatty acids to the nucleic acids may be bound directly or via a linker (a linkage region). In some embodiments, the linker used to bind the fatty acids to the nucleic acids comprises a nucleic acid. In some embodiments, the linker used to bind the fatty acids to the nucleic acids does not comprise a nucleic acid. Exemplary linkers include but are not limited to: —O—P(═O)(OH)—O—, —O—CO—O—, —NH—CO—O—, —NH—CO—NH—, —NH—(CH₂)ₙ₁—, —S—(CH₂)ₙ₁—, —CO—(CH₂)ₙ₁—CO—, —CO—(CH₂)ₙ₁—NH—, —NH—(CH₂)ₙ₁—NH, —CO—NH—(CH₂)ₙ₁—NH—CO—, —C(═S)—NH—(CH₂)ₙ₁—NH—CO—, —C(═S)—NH—(CH₂)ₙ₁—NH—C(═S)—, —CO—O—(CH₂)ₙ₁—O—CO—, —C(═S)—O—(CH₂)ₙ₁—O—CO—, —C(═S)—O—(CH₂)ₙ₁—O—C(═S)—, —CO—NH—(CH₂)ₙ₁—O—CO—, —C(═S)—NH—(CH₂)ₙ₁—O—CO—, —C(═S)—N—H—(CH₂)ₙ₁—O—C(═S)—, —CO—NH—(CH₂)ₙ₁—O—CO—, —C(═S)—NH—(CH₂)ₙ₁—CO—, C(═S)—O—(CH₂)ₙ₁—NH—CO—, —C(═S)—NH—(CH₂)ₙ₁—O—C(═S)—, —NH—(CH₂CH₂O)ₙ₃—CH(CH₂0H)—, —NH—(CH₂CH₂O)ₙ₂—CH₂—, —NH—(CH₂CH₂O)ₙ₂—CO—, —O—(CH₂)ₙ₃—S—S—(CH₂)ₙ₄—O—P(=0)₂—, —CO—(CH₂)ₙ₃—O—CO—NH—(CH₂)ₙ₄—, and —CO—(CH₂)ₙ₃—CO—NH—(CH₂)ₙ₄.

In accordance with the present disclosure, the nucleic acid of a conjugate may be an oligonucleotide (e.g., antisense oligonucleotide (ASO) and CpG oligodeoxynucleotides (CpG ODN)), an aptamer or a variant thereof, an antisense RNA, a small RNA, an RNAi agent, a short interfering nucleic acid (siRNA), a short hairpin RNA (shRNA) molecule, a long non-coding RNA (long ncRNAs, lncRNA), a small activating RNA (SaRNA), a micro-RNA (miRNA), a messenger RNA (mRNA), a double-stranded RNA (dsRNA), a circular RNA and the like. The nucleic acid molecule is a therapeutic nucleic acid.

An “oligonucleotide” refers to a short polymer of nucleotides and/or nucleotide analogs. An oligonucleotide may be 10-200 nucleotides in length, or 10-100 nucleotides in length, Or 10-50 nucleotides in length, or 50-100 nucleotides in length. In some embodiments, the oligonucleotide may comprise at least 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides. A “CpG oligodeoxynucleotide (or CpG ODN)” is a short single-stranded DNA molecule that contains a CpG motif consisting of a cytosine triphosphate deoxynucleotide (“C”) followed by a guanine triphosphate deoxynucleotide (“G”). The “p” refers to the phosphodiester link between consecutive nucleotides. CpG ODNs are a new class of Th-1 type immune stimulant that binds and activates Toll-like receptor 9 (TLR-9). An antisense oligonucleotide (ASO) is a short, synthetic, single-stranded oligodeoxynucleotide that can alter RNA and reduce, restore, or modify protein expression through several distinct mechanisms.

An “RNAi agent” is a nucleic acid molecule including a sequence that recognize a target mRNA sequence to direct target-specific RNA interference (RNAi). An RNAi agent may be a single-stranded oligonucleotide or a double-stranded oligonucleotide. An RNAi agent may be a siRNA (short inhibitory RNA), a shRNA (short or small hairpin RNA), a dsRNA (double-stranded RNA), and a microRNA, etc.

A “small RNA” is a short (about 18 to 30 nucleotides), non-coding RNA molecule that can regulate gene expression in both the cytoplasm and the nucleus via post-transcriptional gene silencing (PTGS), chromatin-dependent gene silencing (CDGS) or RNA activation (RNAa). There are three main classes of small RNAs: microRNAs (miRNAs), siRNAs and Piwi-interacting RNAs (piRNAs) (e.g., reviewed by Farazi et al., the growing catalog of small RNAs and their association with distinct Argonaute/Piwi family members. Development. 2008; 135(7):1201-1214).

A “mRNA” refers to a single stranded RNA molecule that is complementary to one strand of the DNA strands of a gene. It provides the genetic code and template to translate the genetic codes into their corresponding proteins.

A “aptamer” refers to a biomolecule that binds a specific target molecule and modulates the target's activity, structure, or function. An aptamer may be nucleic acid or amino acid based. In the context of the present disclose, an aptamer is a nucleic acid aptamer.

In some embodiments, the nucleic acid of the present conjugate is a therapeutic nucleic acid. As used herein, the term “therapeutic nucleic acid” refers to nucleic acid molecules used as therapeutics. Exemplary therapeutic nucleic acids include aptamers, antisense oligonucleotides, mRNAs, cDNAs, RNAi molecules such as siRNA or shRNA, and saRNAs, and the like.

In some embodiments, the nucleic acid moiety is an activating nucleic acid moiety or an antisense nucleic acid moiety.

In some embodiments, the nucleic acid of the present conjugate may be modified to facilitate enhanced efficacy and specificity, and/or can be modified to improve stability. Various combinations of modifications (e.g., chemical modifications) and/or conjugations may be used to modify the nucleic acid of the present conjugate. Nucleotide analogues may be used to modify the nucleic acid, including sugar- and/or backbone-modified ribonucleotides, e.g., phosphothioate group, sugar-modification at the 2′ OH-group, e.g., 2′-fluoro, 2′-amino and/or 2′-thio modifications. Particularly exemplary modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2-aminopurine, 2′-amino-butyryl-pyrene-uridine, and 2,6-diaminopurine; the 4′ position, e.g., 4-thio-uridine; and/or the 5′ position, e.g., 5-amino-allyl-uridine, 5-bromo-uridine, 5-iodo-uridine, 5-methyl-cytidine, 5-fluoro-cytidine, 5-fluoro-uridine and 5-ribo-thymidine. Additional modified residues include, deoxy-abasic, inosine, N3-methyl-uridine, N6,N6-dimethyl-adenosine, pseudouridine, purine ribonucleoside, ribavirin, locked nucleic acids (LNAs), and/or peptide nucleic acids (PNAs).

### Aptamers

In one preferred embodiment, the nucleic acid of the present conjugate is an aptamer, for example, an aptamer that is identified by SELEX and binds a specific target with high affinity. The target may be a protein, a peptide, a nucleic acid molecule, a lipid, a sugar, a compound, a cell, a tissue, a bacterium, and other analytes.

An aptamer is a biomolecule that binds a specific target molecule and often modulates the target's activity, structure, or function. Aptamers often are referred to as “chemical antibodies,” having similar characteristics as antibodies. An aptamer can be nucleic acid or amino acid based, i.e., either a nucleic acid aptamer or peptide aptamer. Nucleic acid aptamers have specific binding affinity to target molecules through interactions other than classic Watson-Crick base pairing. Nucleic acid aptamers are capable of specifically binding selected targets with high affinity. Some aptamers through binding, can interfere their targets' ability to function.

Aptamers of the present disclosure are synthetic oligonucleotides. A typical nucleic acid aptamer is approximately 10-15 kDa in size, binds its target with nanomolar to sub-nanomolar affinity, and discriminates against closely related targets. A target of a nucleic acid aptamer may be but is not limited to, a protein, a nucleic acid molecule, a peptide, a small molecule and a whole cell.

Nucleic acid aptamers may be ribonucleic acid (RNA), deoxyribonucleic acid (DNA), or mixed ribonucleic acid and deoxyribonucleic acid (DNA/RNA hybrid). Aptamers may be single stranded. A suitable nucleotide length for an aptamer ranges from about 15 to about 150 nucleotides, and in various other preferred embodiments, 15-30 nucleotides, 20-25 nucleotides, 20-45 nucleotides, 30-100 nucleotides, 30-60 nucleotides, 25-70 nucleotides, 25-60 nucleotides, 40-60 nucleotides, 25-40 nucleotides, 30-40 nucleotides, any of 18, 19, 20, 21, 22, 23, 24, 25, 26, 27, 28, 29, 30, 31, 32, 33, 34, 35, 36, 37, 38, 39 or 40 nucleotides, or 30-50 nucleotides, 40-70 nucleotides, or 50-100 nucleotides in length. In some embodiments, an aptamer may be 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length. In other embodiments, an aptamer may be 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length. However, the sequence can be designed with sufficient flexibility such that it can accommodate interactions of aptamers with targets.

The term “nucleotide” refers to the monomer of nucleic acids, a chemical compound comprised of a heterocyclic base, a sugar and one or more phosphate groups. The base is a derivative of purine and pyrimidine and the sugar is a pentose, either deoxyribose or ribose.

As used herein, the term “modification” refers to the technique of chemically reacting a nucleic acid, e.g., an oligonucleotide, with chemical reagents. A nucleic acid may be modified in the base moiety, sugar moiety or phosphate backbone. The modifications include, but are not limited to, 2′-position sugar modifications, 5-position pyrimidine, modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, unusual base-pairing combinations such as the isobases isocytidine and isoguanidine and the like. Modifications can also include 3′ and 5′ modifications such as capping. The nucleic acid molecule may also be modified by conjugation to a moiety having desired biological properties. Such moiety may include, but is not limited to, compounds, peptides and proteins, carbohydrates, antibodies, enzymes, polymers, drugs and fluorophores. In some examples, the polynucleotide is conjugated to a lipophilic compound such as cholesterol, dialkyl glycerol, diacyl glycerol, or a non-immunogenic, high molecular weight compound or polymer such as PEG (polyethylene glycol) or other water soluble pharmaceutically acceptable polymers including, but not limited to, polyaminoamines (PAMAM) and polysaccharides such as dextran, or polyoxazolines (POZ). The modifications may be intended, for example, to increase the in vivo stability of nucleic acid molecules or to enhance or to mediate delivery of the molecules.

Aptamers may be either monovalent or multivalent. Aptamers may be monomeric, dimeric, trimeric, tetrameric or other higher multimeric. Individual aptamer monomers may be linked to form multimeric aptamer fusion molecules. As a non-limiting example, a linking oligonucleotide (i.e., linker) may be designed to contain sequences complementary to both 5′-arm and 3′-arm regions of random aptamers to form dimeric aptamers. For trimeric or tetrameric aptamers, a small trimeric or tetrameric (i.e., a Holliday junction-like) DNA nanostructure will be engineered to include sequences complementary to the 3′-arm regions of the random aptamers, therefore creating multimeric aptamer fusion through hybridization. In addition, 3 to 5 or 5 to 10 dT rich nucleotides can be engineered into the linker polynucleotides as a single stranded region between the aptamer-binding motifs, which offers flexibility and freedom of multiple aptamers to coordinate and synergize multivalent interactions with cellular ligands or receptors. Alternatively, multimeric aptamers can also be formed by mixing biotinylated aptamers with streptavidin.

As used herein, the term “multimeric aptamer” or “multivalent aptamer” refers to an aptamer that comprises multiple monomeric units, wherein each of the monomeric units can be an aptamer on its own. Multivalent aptamers have multivalent binding characteristics. A multimeric aptamer can be a homomultimer or a heteromultimer. The term “homomultimer” refers to a multimeric aptamer that comprises multiple binding units of the same kind, i.e., each unit binds the same binding site of the same target molecule. The term “heteromultimer” refers to a multimeric aptamer that comprises multiple binding units of different kinds, i.e., each binding unit binds a different binding site of the same target molecule, or each binding unit binds a binding site on different target molecule. Thus, a heteromultimer can refer to a multimeric aptamer that binds one target molecule at different binding sites or a multimeric aptamer that binds different target molecules. A heteromultimer that binds different target molecules can also be referred to as a multi-specific multimer.

Nucleic acid aptamers comprise a series of linked nucleosides or nucleotides. The term “nucleic acid,” in its broadest sense, includes any compound and/or substance that comprise a polymer of nucleotides. These polymers are often referred to as polynucleotides. Exemplary nucleic acid molecules or polynucleotides of the invention include, but are not limited to, either D- or L-nucleic acids, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic acid aptamers may be ribonucleic acid, deoxyribonucleic acid, or mixed ribonucleic acid and deoxyribonucleic acid. Aptamers may be single stranded ribonucleic acid, deoxyribonucleic acid or mixed ribonucleic acid and deoxyribonucleic acid.

Aptamers can be generated against a target molecule (e.g., a protein of interest) using a process called either in vitro selection (Ellington and Szostak; In vitro selection of RNA molecules that bind specific ligands. Nature. 1990; 346: 818-822) or SELEX (Tuerk and Gold, Systematic evolution of ligands by exponential enrichment: RNA ligands to bacteriophage T4 DNA polymerase; Science, 1990, 249: 505-510). This method allows the in vitro evolution of nucleic acid molecules with highly specific binding target molecules. The SELEX method is described in, for example, U.S. Pat. Nos. 7,087,735, 5,475,096 and 5,270,163; the contents of each of which are incorporated herein by reference in their entirety. Nucleic acid aptamers can be synthesized using methods well-known in the art. For example, the disclosed aptamers may be synthesized using standard oligonucleotide synthesis technology known in the art.

In some embodiments, the aptamer comprises at least one chemical modification. In some embodiments, the chemical modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate backbone. In a preferred embodiment, the high molecular weight, non-immunogenic compound is polyalkylene glycol, and more preferably is polyethylene glycol (PEG). The process of covalent conjugation of PEG to another molecule, normally a drug or therapeutic protein is known as PEGylation. PEGylation is routinely achieved by incubation of a reactive derivative of PEG with the target molecule. The covalent attachment of PEG to a drug or therapeutic protein can mask the agent from the host's immune system, thereby providing reduced immunogenicity and antigenicity, and increase the hydrodynamic size (size in solution) of the agent which prolongs its circulatory time by reducing renal clearance. PEGylation can also provide water solubility to hydrophobic drugs and proteins.

In some embodiments, nucleic acid aptamers are provided in which the P(O)O group is replaced by P(O)S (“thioate”), P(S)S (“dithioate”), P(O)NR2 (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the nucleic acid aptamers are required to be identical.

As non-limiting examples, a nucleic acid aptamer can include D-ribose or L-ribose nucleic acid residues and can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, an inverted deoxynucleoside or inverted ribonucleoside, a 2′-deoxy-2′-fluoro-modified nucleoside, a 2′-amino-modified nucleoside, a 2′-alkyl-modified nucleoside, a morpholino nucleoside, a phosphoramidate or a non-natural base comprising nucleoside, or any combination thereof. Alternatively, a nucleic acid aptamer can comprise at least two modified ribonucleosides, at least 3, at least 4, at least 5, at least 6, at least 7, at least 8, at least 9, at least 10, at least 15, at least 20 or more modified ribonucleosides, up to the entire length of the molecule. The modifications need not be the same for each of such a plurality of modified deoxy- or ribonucleosides in a nucleic acid molecule.

An aptamer of the present invention may include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, those disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993.

In some embodiments, the nucleic acid aptamer comprises one or more regions of double-stranded character. Such double stranded regions may arise from internal self-complementarity or complementarity with a second or further aptamers or oligonucleotide molecule. In some embodiments, the double stranded region may be from 4-12, 4-10, 4-8 base pairs in length. In some embodiments, the double stranded region may be 5, 6, 7, 8, 9, 10, 11 or 12 base pairs. In some embodiments, the double stranded region may form a stem region. Such extended stem regions having double stranded character can serve to stabilize the nucleic acid aptamer. As used herein, the term “double stranded character” means that over any length of two nucleic acid molecules, their sequences form base pairings (standard or nonstandard) of more than 50 percent of the length.

Aptamers may be further modified to provide protection from nuclease and other enzymatic activities. The aptamer sequence can be modified by any suitable methods known in the art. For example, phosphorothioate can be incorporated into the backbone, and 5′-modified pyrimidine can be included in 5′ end of ssDNA for DNA aptamers. For RNA aptamers, modified nucleotides such as substitutions of the 2′—OH groups of the ribose backbone, e.g., with 2′-deoxy-NTP or 2′-fluoro-NTP, can be incorporated into the RNA molecule using T7 RNA polymerase mutants. The resistance of these modified aptamers to nuclease can be tested by incubating them with either purified nucleases or nuclease from mouse serum, and the integrity of aptamers can be analyzed by gel electrophoresis.

In some embodiments, such modified nucleic acid aptamers may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modifications. For example, all purine nucleotides may have one type of modification (or are unmodified), while all pyrimidine nucleotides have another, different type of modification (or are unmodified). In this way, oligonucleotides, or libraries of oligonucleotides are generated using any combination of modifications as disclosed herein.

According to certain embodiments of the present invention, variants and derivatives of aptamers are provided. The term “derivative” is used synonymously with the term “variant” and refers to a molecule that has been modified or changed in any way relative to a reference or starting aptamer. The nucleic acid sequence of aptamer variants may possess substitutions, deletions, and/or insertions at certain positions within the nucleotide sequence, as compared to a reference or starting sequence. Ordinarily, variants will possess at least about 50% identity (homology) to a reference sequence, and preferably, they will be at least about 80%, more preferably at least about 90% identical (homologous) to a reference sequence.

In some embodiments, the aptamer is modified with a functional group that can be used to covalently bind to a fatty acid moiety. The functional group can be attached to the 3′ or 5′ end of the aptamer. In some embodiments, an additional linker group or nucleotide replacement may be added to the aptamer for conjugation.

In some embodiments, the 5′ end of the aptamer has a Dibenzocyclooctyne (DBCO) group:

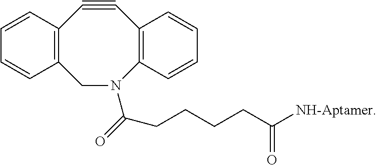

The DBCO group can undergo copper-free Click Chemistry reactions with an azide group.

In some embodiments, variant mimics of aptamers of the present disclosure are provided. As used herein, the term “variant mimic” is one which contains one or more nucleic acids which would mimic an activated sequence. The nucleic acid sequences of variant mimics may comprise naturally occurring nucleic acids, or alternatively, non-naturally occurring nucleic acids.

### Fatty Acid Moiety

In accordance with the present disclosure, a fatty acid moiety is conjugated to a nucleic acid, such as an aptamer. The fatty acid moiety can be a native fatty acid or a fatty acid like molecule.

In some embodiments, the molecular weight of the fatty acid or the fatty acid moiety is at least 500 Da, such as between 500-1K Da, between 1K-1.5K Da, between 1.5K-2K Da, between 2K-2.5K Da, or between 2.5K-3K Da.

A fatty acid is a carboxylic acid with a typical RCOOH structure consisting of a methyl end (—CH3), a hydrocarbon chain (R) (also called “aliphatic tail”) and a terminal carboxyl group (—COOH). The hydrocarbon chain may comprise 4-30 carbon atoms, may be saturated or unsaturated (with at least one double bond or triple bond), and may have a straight or branched hydrocarbon chain. They are often indicated as a schematic formula as in CN:p n-x where CN represents total number of cardon atoms, p is the number of double bonds and x indicates position of the first double bond from the methyl end (n) (IUPAC-IUB Commission, Eur J Biochem 1977, 79:11-21). In some embodiments, a fatty acid consists of a straight chain of an even number of carbon atoms, with hydrogen atoms along the length of the chain and at one end of the chain and a carboxyl group (—COOH) at the other end.

Fatty acids can be divided into several groups with respect to their structure, physiological role and biological effects. Fatty acids can be classified as saturated and unsaturated fatty acids according to their structures. A fatty acid-like molecule refers to a modified fatty acid in which certain carbon atoms may be replaced by other atoms or groups of atoms and which may be substituted. The fatty acid-like molecules are modified in order to better facilitate conjugating strategies. As a non-limiting example, a native fatty acid may be modified to contain an amino terminus. In other examples, a fatty acid molecule may comprise one or more hydrophilic groups to facilitate the conjugation reaction.

In some embodiments, the fatty acid moiety comprises an alkyl, alkenyl, alkynyl, aryl, or aralkyl group. This alkyl, alkenyl, alkyl, aryl, or aralkyl group may comprise about 10, 11, 12, 13, 14, 15, 16, 17, 18, 19, 20, 21, 22, 23, 24, 25, 26, 27, 28, 29, 30 carbon atoms or more.

In some embodiments, the alkyl group comprises about 10, 11, 12, 13, 14, 15, 16, 17, 18, 19, 20, 21, 22, 23, 24, 25, 26, 27, 28, 29, or 30 carbon atoms or more.

In certain embodiments, the fatty acid moiety region may comprise a saturated or unsaturated, linear or branched, substituted or unsubstituted aliphatic chain.

In some embodiments, the fatty acid moiety region may contain one or more unsaturated carbon bonds. In some embodiments, the unsaturated bonds are all contained within the same chain.

As used herein, the term “alkyl” may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 2-100 carbon atoms, or 2-50 carbon atoms, or 10-50 carbon atoms, or 2-30 carbon atoms, or 10-30 carbon atoms, or 12-20 carbon atoms.

As used herein, the term “alkenyl” refers to a branched or unbranched hydrocarbon having at least one carbon-carbon double bond.

As used herein, the term “alkynyl” refers to a branched or unbranched hydrocarbon having at least one carbon-carbon triple bond.

As used herein, the term “aryl”, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic.

In some embodiments, a fatty acid moiety comprises 10, 11, 12, 13, 14, 15, 16, 17, 18, 19, 20, 21, 22, 23, 24, 25, 26, 27, 28, 29, 30 or more carbon atoms. In some embodiments, a fatty acid moiety comprises 10 or more carbon atoms. In some embodiments, a fatty acid moiety comprises 11 or more carbon atoms. In some embodiments, a fatty acid moiety comprises 12 or more carbon atoms. In some embodiments, a fatty acid moiety comprises 13 or more carbon atoms. In some embodiments, a fatty acid moiety comprises 14 or more carbon atoms. In some embodiments, a fatty acid comprises 15 or more carbon atoms. In some embodiments, a fatty acid moiety comprises 16 or more carbon atoms. In some embodiments, a fatty acid moiety comprises 17 or more carbon atoms. In some embodiments, a fatty acid moiety comprises 18 or more carbon atoms. In some embodiments, a fatty acid moiety comprises 19 or more carbon atoms. In some embodiments, a fatty acid moiety comprises 20 or more carbon atoms. In some embodiments, a fatty acid moiety comprises 25 or more carbon atoms. In some embodiments, a fatty acid moiety comprises 30 or more carbon atoms.

In some embodiments, the fatty acid moiety comprises —(CH₂)ₐ—COOH, wherein a=12-26 In some embodiments, a=16. In other embodiments, a=18.

In some embodiments, the fatty acid moiety comprises at least one ethylene glycol group: —(OCH₂CH₂)—. In some embodiments, the fatty acid moiety comprises between 1 and 10 ethylene glycol groups. In some embodiments, the fatty acid moiety comprises 1, 2, 3, 4, or 5 ethylene glycol groups.

In some embodiments, the fatty acid moiety comprises at least one polyethylene glycol (PEG) group: —(OCH₂CH₂)_{b}—, wherein b=1-10. In some embodiments, b=4.

In some embodiments, the fatty acid moiety comprises at least one amide group (—NH—CO—).

In some embodiments, the fatty acid moiety comprises at least one —(OCH₂CH₂—OCH₂CH₂—NH—CO)— group. In some embodiments, the fatty acid moiety comprises 1, 2, 3, 4, or 5 —(OCH₂CH₂—OCH₂CH₂—NH—CO)— groups.

In some embodiments, the fatty acid moiety comprises at least one —(CO—CH₂—OCH₂CH₂—OCH₂CH₂—NH)— group. In some embodiments, the fatty acid moiety comprises between 1 and 10 —(CO—CH₂—OCH₂CH₂—OCH₂CH₂—NH)— groups. In some embodiments, the fatty acid moiety comprises 2 —(CO—CH₂—OCH₂CH₂—OCH₂CH₂—NH)— groups.

In some embodiments, the fatty acid moiety comprises at least an amino acid group, or a derivative/analog thereof. In some embodiments, the fatty acid moiety comprises at least one glutamic acid group, or a derivative/analog thereof, such as

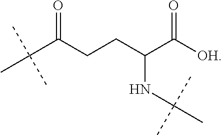

In some embodiments, the glutamic acid group is a 7-glutamic acid group

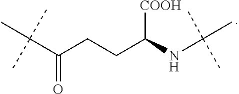

In some embodiments, the fatty acid moiety comprises a piperazine group. In some embodiments, the fatty acid moiety comprises piperazine-2-carboxylic acid group

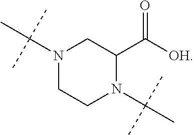

In some embodiments, the fatty acid moiety comprises a

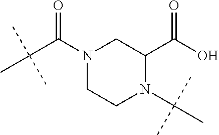

group. In some embodiments, the fatty acid moiety comprises a

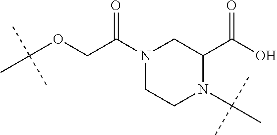

group.

In some embodiments, the fatty acid moiety comprises an azide (—N₃) group.

In some embodiments, the fatty acid moiety comprises a general formula of:

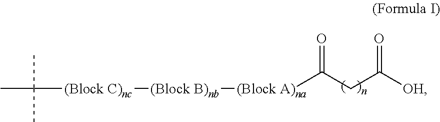

wherein n is an integer from 12 to 26,
Block A is

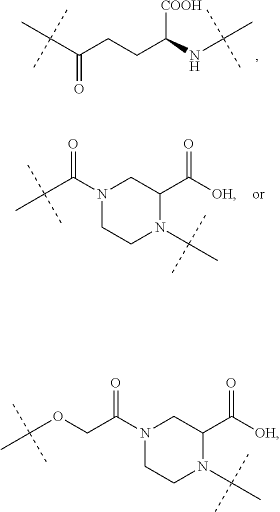

na is 0 or 1,
Block B is —(OCH₂CH₂)— or —(CO—CH₂—OCH₂CH₂—OCH₂CH₂—NH)—,
nb is an integer from 1 to 10,
Block C is

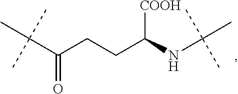

and
nc is 0 or 1.

In some embodiments, the fatty acid moiety comprises a general formula of:

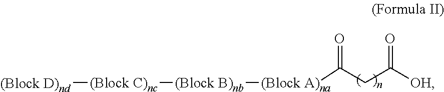

wherein n is an integer from 12 to 26,
Block A is

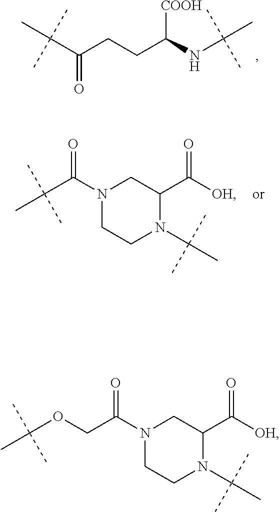

na is 0 or 1,
Block B is —(OCH₂CH₂)— or —(CO—CH₂—OCH₂CH₂—OCH₂CH₂—NH)—,
nb is an integer from 1 to 10,
Block C is

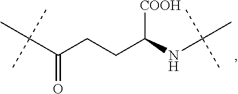

nc is 0 or 1,
Block D comprise a functional group that can covalently bind to a nucleic acid, and
nd is 0 or 1.

In some embodiments, Block D comprises an azide group, an alkyne group, a hydroxy group, a sulfhydryl group, or an amino group. In some embodiments, Block D is R_{d}—N₃, wherein R_{d} comprises an alkyl group, an amino alkyl group, an amine group, and/or an alkoxyl group. In some embodiments, Block D is

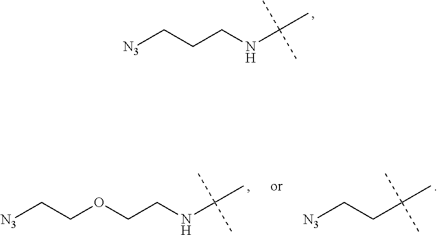

In some embodiments, the fatty acid moiety comprises —(CH₂)₁₆—COOH. In some embodiments, the fatty acid moiety further comprises at least one —(OCH₂CH₂—OCH₂CH₂—NH—CO—CH₂)— group. In some embodiments, the fatty acid moiety further comprises at least one γ-glutamic acid group. Non-limiting examples include
GTFA-1 having a structure of:

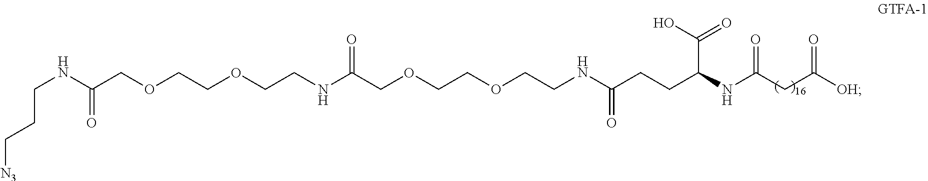

GTFA-1′ having a structure of

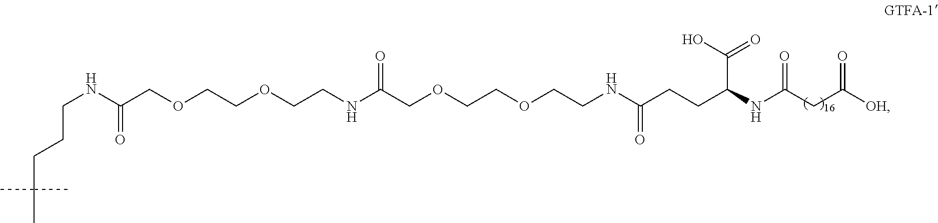

GTFA-3 having a structure of:

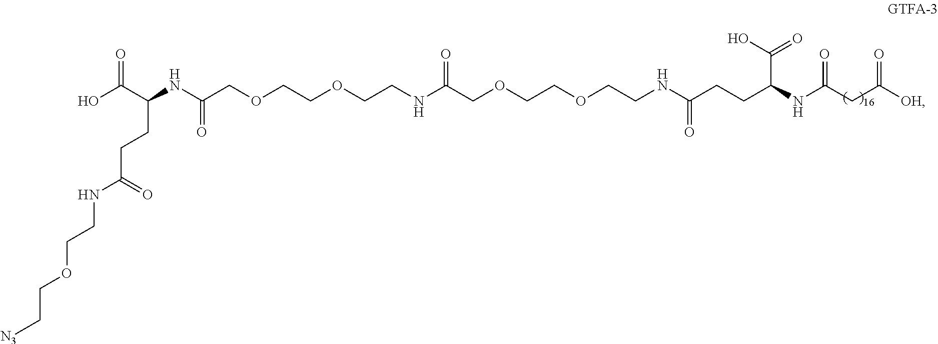

or
and GTFA-3′ having a structure of

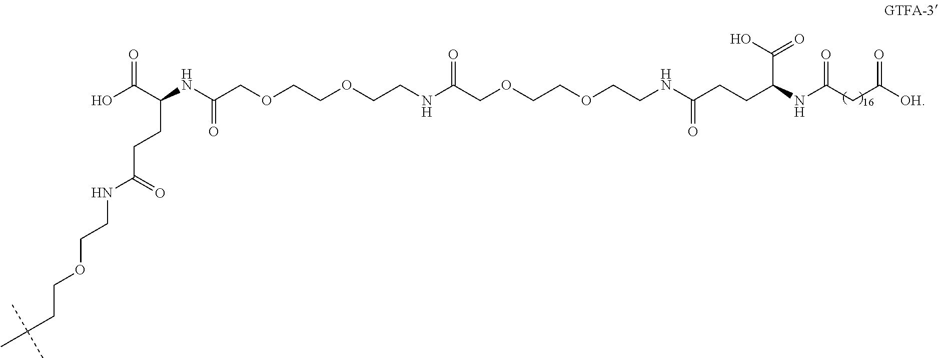

In some embodiments, the fatty acid moiety comprises —(CH₂)₁₆—COOH. In some embodiments, the fatty acid moiety further comprises one polyethylene glycol (PEG) group. In some embodiments, the fatty acid moiety further comprises piperazine-2-carboxylic acid group. Non-limiting examples include GTFA-2 having a structure of:

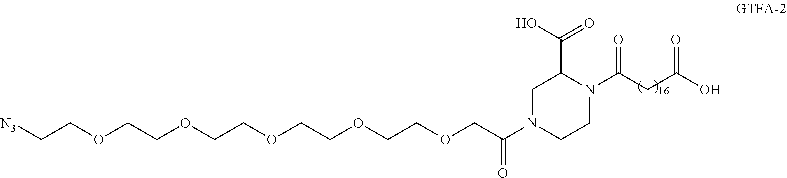

or
GTFA-2′ having a structure of

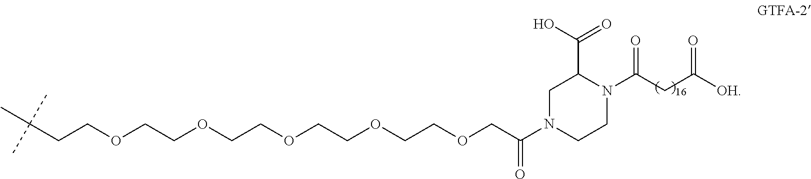

Synthesis of Fatty acid conjugates

Nucleic acid molecules disclosed herein encompass native and synthetic or modified nucleic acids. A modified nucleic acid has one or more modifications, e.g., a base modification, a backbone modification, etc., to provide the nucleic acid with a new or enhanced feature (e.g., improved stability). The nucleic acid molecules and the fatty acids can be synthesized with any suitable method known in the art. The nucleic acids and the fatty acids can then be attached via any suitable method known in the art. In a non-limiting example, the nucleic acids and the fatty acids are attached via click chemistry, such as an azide-alkyne cycloaddition.

The term “click chemistry,” encompasses various reactions that are selective and proceed with high yields with little to no byproducts under simple reaction conditions and solvents. As used herein, the term “click chemistry” refers to the Huisgen cycloaddition or the 2,3-dipolar cycloaddition between an azide and a terminal alkyne to form a 1,2,4-triazole. The term “cycloaddition” as used herein refers to a chemical reaction in which two or more 71-electron systems (e.g., unsaturated molecules or unsaturated parts of the same molecule) combine to form a cyclic product in which there is a net reduction of the bond multiplicity. In a cycloaddition, the π electrons are used to form new sigma bonds. The product of a cycloaddition is called an “adduct” or “cycloadduct”. Different types of cycloadditions are known in the art including, but not limited to, [3+2] cycloadditions and Diels-Alder reactions. [3+2] cycloadditions, which are also called 2,3-dipolar cycloadditions, occur between a 1,3-dipole and a dipolarophile and are typically used for the construction of five-membered heterocyclic rings. The terms “[3+2] cycloaddition” also encompasses “copperless” [3+2]cycloadditions between azides and cyclooctynes and difluorocyclooctynes described by Bertozzi et al. J. Am. Chem. Soc., 2004, 126:15046-15047. Any reagent that can be used to facilitate the Huisgen cycloaddition can be used as click chemistry reagent. In some embodiments, the click chemistry reagent comprises pyridyl azide. In some embodiments, the click chemistry reagent comprises picolyl azide. Without limitation, any isomer of picolyl azide can be used.

In some embodiments, the fatty acid moiety is covalently conjugated to the 5′ end of the nucleic acid sequence. In other embodiments, the fatty acid moiety is covalently conjugated to the 3′ end of the nucleic acid sequence.

In some embodiments, the conjugates have a general formula of

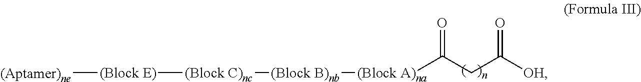

wherein n is an integer from 12 to 26,
Block A is

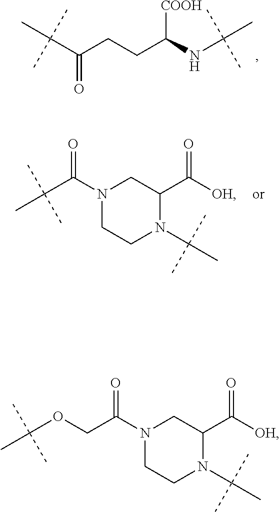

na is 0 or 1,
Block B is —(OCH₂CH₂)— or —(CO—CH₂—OCH₂CH₂—OCH₂CH₂—NH)—,
nb is an integer from 1 to 10,
Block C is

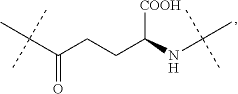

nc is 0 or 1,
Block E is an optional linker,
ne is an integer from 1 to 10.

In some embodiments, Block E is a group that is formed by a Click Chemistry reaction between DBCO and azide. In some embodiments, Block E comprises

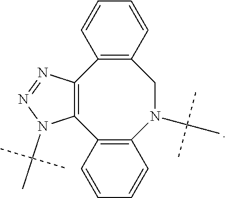

In some embodiments, a fatty acid-nucleic acid conjugate may be generated following the general steps of 1) modifying the nucleic acid to add an active group. such as addition of a DBCO group at one end of the nucleotide sequence; 2) synthesizing a fatty acid moiety with one or more functional groups, for example fatty acid moieties discussed in Example 1 of the present disclosure; and 3) synthesizing the fatty acid-nucleic acid conjugate through the chemical reaction between the functional groups of the fatty acid moiety and the nucleic acid. The reaction mixture may be further processed to isolate the fatty acid-nucleic acid conjugate.

In some embodiments, a fatty acid conjugation of an anti-VWF aptamer is provided by the present disclosure. The anti-VWF aptamer may comprise a polynucleotide sequence: NH2-mGmCmCmAmGmGmGmAmCmCmUmAmAmGmAmCmAmCmAmUmGmUmCmCmC mUmGmGmC-idT (SEQ ID NO: 1, BT100), wherein “NH” is a 5′-hexylamine linker phosphoramidite, and wherein “idT” is an inverted deoxythymidine, and wherein “mN” is a 2′-O-Methyl containing residue. As a non-limiting example, the fatty acid conjugation of BT100 comprises a polynucleotide sequence: (5′-)Palmitic acid-linker-mGmCmCmAmGmGmGmAmCmCmUmAmAmGmAmCmAmCmAmUmGmUmCmCmC mUmGmGmCidT (-3′) (SEQ ID NO: 3, BT500).

As another example, the fatty acid conjugation of BT100 comprises a polynucleotide sequence: (5′-)GTFA-1-linker-mGmCmCmAmGmGmGmAmCmCmUmAmAmGmAmCmAmCmAmUmGmUmCmCmC mUmGmGmCidT (-3′) (SEQ ID NO: 4, BT600).

As another example, the fatty acid conjugation of BT100 comprises a polynucleotide sequence: (5′-) GTFA-3-linker-mGmCmCmAmGmGmGmAmCmCmUmAmAmGmAmCmAmCmAmUmGmUmCmCmC mUmGmGmCidT (-3′) (SEQ ID NO: 5, BT700).

### Pharmaceutical Compositions and Methods of Administrations

In another aspect, the present disclosure provides a pharmaceutical composition including a nucleic acid-fatty acid conjugate of the present disclosure. The nucleic acid of the conjugate is a therapeutic nucleic acid. In some embodiments, the pharmaceutical composition further comprises at least one pharmaceutically acceptable carrier, diluent or excipient.

The composition may be formulated for particular routes of administration such as parental administration, enteral administration, oral administration, or other appropriate routes. Parental administration may be performed by injection, or by the insertion of an indwelling catheter, including but not limited to intravenous (IV), intramuscular (IM), subcutaneous (SC), epicutaneous injection, peridural injection, intracerebral (into the cerebrum) administration, intracerebroventricular (into the cerebral ventricles) administration, extra-amniotic administration, nasal administration, intra-arterial, intracardiac, intraosseous infusion (IO), intraperitoneal infusion or injection, transdermal diffusion, enteral and gastrointestinal routes, topical administration and oral routes.

In addition, the pharmaceutical compositions of the present disclosure may be made up in a solid form including without limitation capsules, tablets, pills, granules, lyophilizates, powders or suppositories, or in a liquid form including without limitation solutions, suspensions or emulsions. The pharmaceutical compositions can be subjected to conventional pharmaceutical operations such as aseptic manufacturing, sterilization and/or can contain conventional inert diluents, cake forming agents, tonicity agents, lubricating agents, or buffering agents, as well as adjuvants, such as preservatives, stabilizers, wetting agents, emulsifers and buffers, etc.

In some embodiments, pharmaceutical compositions suitable for injectable use typically include sterile aqueous solutions (where water soluble) or dispersions and sterile powders for the extemporaneous preparation of sterile injectable solutions or dispersion.

For intravenous administration, suitable carriers include physiological saline, bacteriostatic water or phosphate buffered saline (PBS). In all cases, the composition is sterile for injection. Preferred pharmaceutical formulations are stable under the conditions of manufacture and storage. In general, the relevant carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, amino acids, sorbitol, sodium chloride in the composition. Prolonged absorption of the injectable compositions can be brought about by including in the composition an agent which delays absorption, for example, aluminum monostearate and gelatin. In some embodiments, a multifunctional excipient such as recombinant albumin may be incorporated into the formulation process to facilitate the stabilization of the conjugate product from degradation or aggregation, to improve solubility and assist in the administration and release of the active component. (BioPharm International, 2012, 23(3):40-44).

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers.

Sterile injectable solutions can be prepared by incorporating the active compound in the required amount in an appropriate solvent with one or a combination of ingredients enumerated above, as required, followed by filtration sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the compositions are preferably delivered in the form of an aerosol spray from pressured container or dispenser which contains a suitable propellant, e.g., a gas such as carbon dioxide, or a nebulizer. It is noted that the lungs provide a large surface area for systemic delivery of therapeutic agents.

In some embodiments, the pharmaceutical composition is formulated for systemic administration, e.g., by intravenous, transmucosal or transdermal means.

Suitable compositions for transdermal application include an effective amount of a conjugate of the present disclosure with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time and means to secure the device to the skin.

In certain embodiments, the pharmaceutical composition is for subcutaneous administration.

### Methods of Use

In another aspect, provided herein is a method for treating a disease or disorder in a patient in need of such treatment, comprising administering to the patient a conjugate of the present disclosure.

Fatty acid conjugated nucleic acids and compositions comprising fatty acid conjugated nucleic acids can be used in a variety of different pharmaceutical, therapeutic, diagnostic and biomedical applications. The present fatty acid modifications improve the potency and efficacy of nucleic acids (e.g., aptamers), and uncover for the potential for oral delivery as well as enhance subcutaneous and intravenous administration. The present fatty acid conjugations may increase potency of therapeutic nucleic acids to allow administration of lower doses, which reduces the potential for toxicity and immunogenicity. The present fatty acid conjugations may increase the stability which allows for less frequent dosing.

As non-limiting examples, the fatty acid conjugated nucleic acids and compositions comprising fatty acid conjugated nucleic acids find use in therapeutic applications.

Non-limiting examples of disease or disorders include, an angiogenic disorder, a cardiovascular disorder, stroke, a neurodegenerative disorder, cancer, a genetic disorder, and an orphan disease, etc.

In some embodiments, the present treatment method may alter onset of symptoms of the disease or disorder.

In one aspect, the present disclosure provides a method for preventing in a subject, a disease or disorder as described above, by administering to the subject a therapeutically effective amount of a conjugate of the present disclosure. Subjects at risk for the disease can be identified by, for example, any or a combination of diagnostic or prognostic assays. Administration of a prophylactic agent can occur prior to the manifestation of symptoms characteristic of the disease or disorder, such that the disease or disorder is prevented or, alternatively, delayed in its progression.

In one embodiment, a subject is administered an initial dose, and one or more maintenance doses of the conjugates of the present disclosure. The maintenance dose or doses are generally lower than the initial dose, e.g., one-half less of the initial dose. A maintenance regimen can include treating the subject with a dose or doses ranging from 0.01 mg to 100 mg/kg of body weight per day, e.g., 100, 10, 1, 0.1, 0.01, 0.001, or 0.00001 mg per kg of body weight per day. The maintenance doses are preferably administered no more than once every 2, 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

In some embodiments, a fatty acid conjugation of BT100 (e.g., BT500, BT600 and BT700) can be used to bind VWF antigen. In some embodiments, the conjugate can be used for treating and/or preventing stroke and transient ischemic attack (TIA), and stroke recurrence in patients with primary ischemic stroke and TIA. For example, the treatment and prevention methods use BT500, BT600 or BT700, in combination with one or more anti-thrombotic drugs.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

The following examples are offered by way of illustration and not by way of limitation.

### Example 1: Methods of Synthesis of Exemplary Fatty Acid Moieties

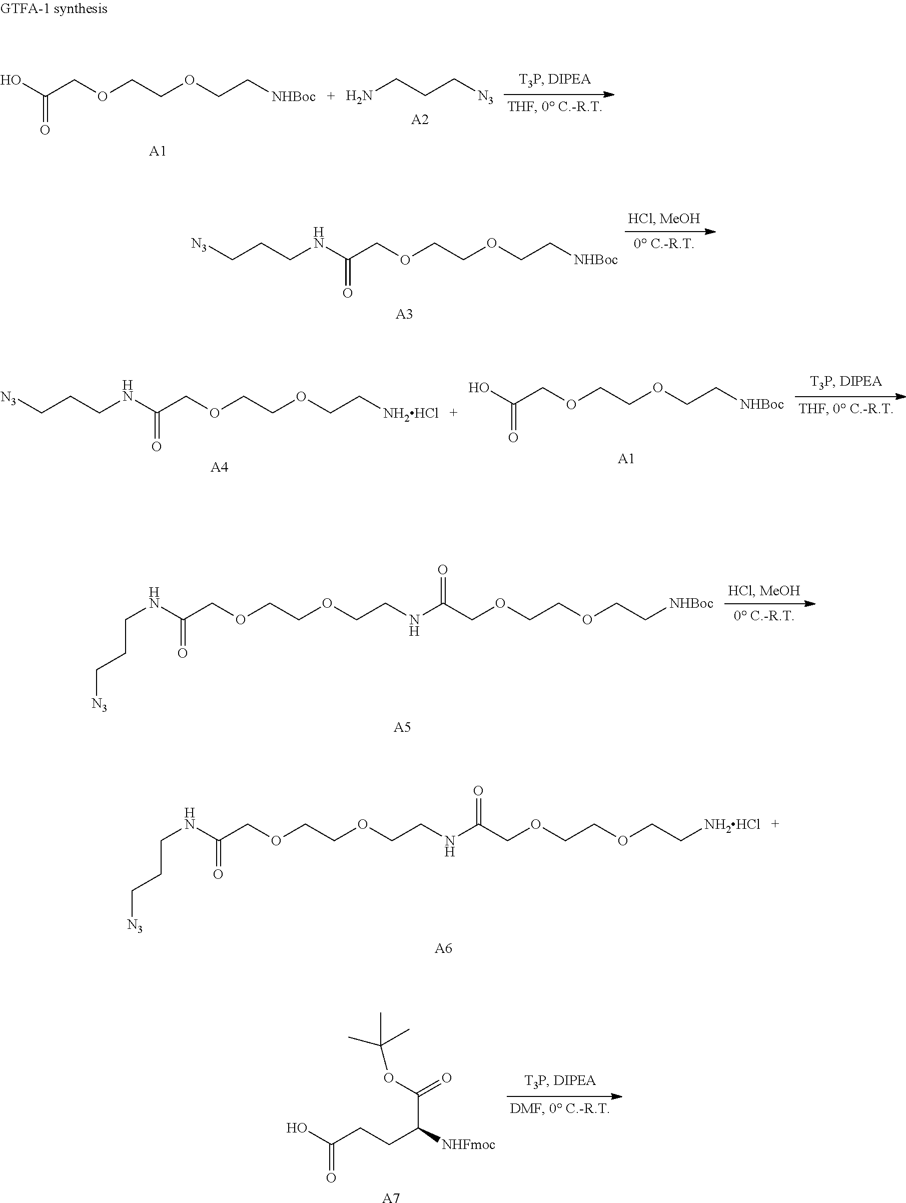

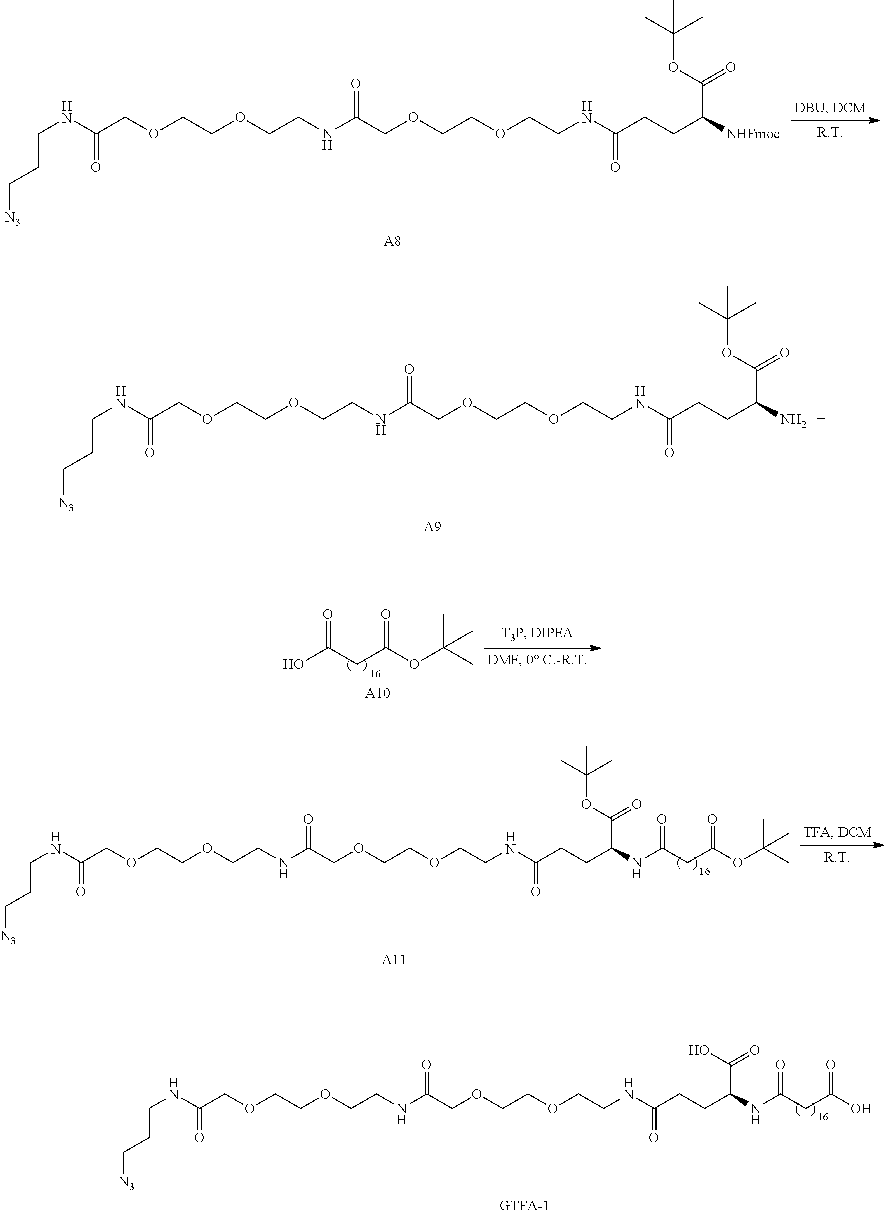

### Step 1:

To a solution of compound A1 (2.46 g, 9.4 mmol) and compound A2 (940 mg, 9.4 mmol) in anhydrous THF (25 ml) was added T₃P (9 ml, 50% in EA, 14 mmol) and DIPEA (3.3 ml, 18.8 mmol) at 0° C. The reaction mixture was stirred for 1 hour and diluted with EtOAc. The mixture was washed with water, brine, dried over Na₂SO₄ and concentrated. The residue was purified by prep-HPLC to give compound A3 (2.8 g, 8.1 mmol). LCMS: 346 [M+H]⁺.

### Step 2:

Compound A3 (2.7 g, 7.8 mmol) was dissolved in 50 ml methanol and HCl (20 ml, 4 N in dioxane, 80 mmol) was added at 0° C. The reaction mixture was stirred at room temperature for 3 hours and concentrated to give compound A4 (2.2 g, 7.8 mmol). LCMS: 246 [M+H]⁺.

### Step 3:

To a solution of compound A4 (1.5 g, 5.2 mmol) and compound A1 (1.4 g, 5.3 mmol) in anhydrous THF (30 ml) was added T₃P (4.6 ml, 50% in EA, 7.8 mmol) and DIPEA (3.6 ml, 20.8 mmol) at 0° C. The reaction mixture was stirred for 1 hour and diluted with EtOAc. The mixture was washed with water, brine, dried over Na₂SO₄ and concentrated. The residue was purified by prep-HPLC to give compound A5 (2.2 g, 4.5 mmol). LCMS: 491 [M+H]⁺.

### Step 4:

Compound A5 (2.2 g, 4.5 mmol) was dissolved in 30 ml methanol and HCl (12 ml, 4 N in dioxane, 48 mmol) was added at 0° C. The reaction mixture was stirred at room temperature for 4 hours and concentrated to give compound A6 (1.7 g, 4 mmol). LCMS: 391 [M+H]⁺.

### Step 5:

To a solution of compound A6 (1.4 g, 3.3 mmol) and compound A7 (1.1 g, 2.6 mmol) in anhydrous DMF (15 ml) was added T₃P (3 ml, 50% in EA, 5 mmol) and DIPEA (2.3 ml, 13.2 mmol) at 0° C. The reaction mixture was stirred for 1.5 hours and diluted with EtOAc. The mixture was washed with water, brine, dried over Na₂SO₄ and concentrated to give crude compound A8 (2.3 g) which was used for next step without further purification. LCMS: 798 [M+H]⁺.

### Step 6:

To a solution of Compound A8 (2.3 g) in 30 ml DCM was added DBU (1 mL). The resulting mixture was stirred at R.T. for 2 hours, then diluted with DCM (100 ml), washed with water (50 mL*2). The organic phase was concentrated, and the residue was dissolved with water (20 mL) and extracted with MTBE (50 mL). The aqueous phase was purified by prep-HPLC to give compound 9 (1.2 g, 2.1 mmol). LCMS: 576 [M+H]⁺.

### Step 7:

To a solution of compound A9 (1.0 g, 1.74 mmol) and compound A10 (640 mg, 1.73 mmol) in anhydrous DMF (10 ml) was added T₃P (1.5 ml, 50% in EA, 2.6 mmol) and DIPEA (605μ, 3.48 mmol) at 0° C. The reaction mixture was stirred for 1 hour and diluted with EtOAc. The mixture was washed with water, brine, dried over Na₂SO₄ and concentrated. The residue was purified by prep-HPLC to give compound 11 (1.4 g, 1.5 mmol). LCMS: 928 [M+H]⁺.

### Step 8:

Compound A11 (1.4 g, 1.5 mmol) was dissolved in 10 ml DCM and TFA (5 ml) was added at 0° C. The reaction mixture was stirred at room temperature for 3 hour and concentrated. The residue was purified by prep-HPLC to give final product GTFA-1 (850 mg, 1.04 mmol). C38H69N7O12 LCMS: 816 [M+H]⁺. ¹H NMR (400 MHz, DMSO-d₆) δ: 13.00-11.00 (br, 2H), 8.00-8.02 (m, 1H), 7.88-7.85 (m, 1H), 7.77-7.74 (m, 1H), 7.66-7.63 (m, 1H), 4.15-4.09 (m, 1H), 3.88 (s, 2H), 3.86 (s, 2H), 3.59-3.51 (m, 8H), 3.44-3.43 (m, 2H), 3.41-3.38 (m, 2H), 3.33-3.25 (m, 4H), 3.21-3.13 (m, 4H), 2.17-2.06 (m, 6H), 1.95-1.88 (m, 1H), 1.75-1.70 (m, 1H), 1.68-1.63 (m, 2H), 1.47-1.45 (m, 4H), 1.22 (s, 24H).

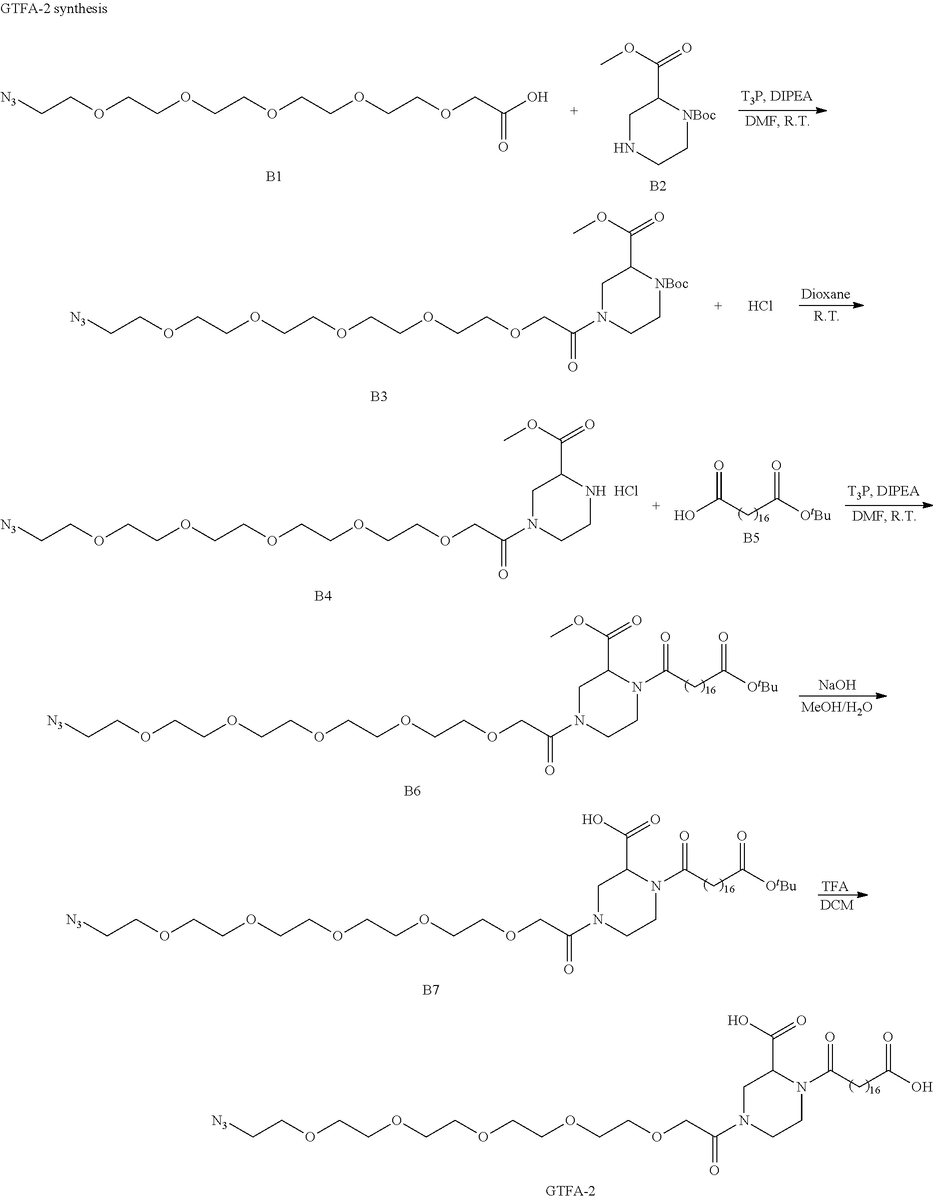

### Step 1:

To a solution of compound 3 (00 mg, 0.5 mmol) and compound B2 (150 mg, 0.6 mmol) in anhydrous DMF (4 ml) was added T₃P (500 μl, 50% in LA, 0.84 mmol) and DIPEA (260 al, 1.5 mmol) at 0° C. The reaction mixture was stirred for 30 minutes and diluted with EtOAc. The mixture was washed with water, brine, dried over Na₂SO₄ and concentrated. The residue was purified by prep-HPLC to give compound B3 (200 mg, 0.37 mmol). LCMS: 548 [M+H].

### Step 2:

Compound 3 (200 mg, 0.37 mmol) was dissolved in 2 ml dioxane and HCl (2 ml, 4 N in dioxane, 8 mmol) was added. The reaction mixture was stirred at room temperature for 40 minutes and concentrated to give compound B4 (180 mg, 0.36 mmol). LCMS: 448 [M+H]⁺.

### Step 3:

To a solution of compound 4 (180 mg, 0.36 mmol) and compound B5 (135 mg, 0.36 mmol) in anhydrous DMF (4 ml) was added T₃P (400 μl, 50% in EA, 0.67 mmol) and DIPEA (260 μl, 1.5 mmol) at 0° C. The reaction mixture was stirred for 1 hour and diluted with EtOAc. The mixture was washed with water, brine, dried over Na₂SO₄ and concentrated. The residue was purified by prep-HPLC to give compound B6 (135 mg, 0.17 mmol). LCMS: 800 [M+H]⁺.

### Step 4:

Compound B6 (135 mg, 0.17 mmol) was dissolved in 4 ml MeOH and NaOH (1 ml, 4 N in water, 4.0 mmol) was added. The reaction mixture was stirred at room temperature for 1 hours and removed MeOH under reduced pressure. The residue was diluted with water and cooled down to 0° C., HCl (2 ml, 2 N, 4.0 mmol) was added. The mixture was extracted with DCM, dried over Na₂SO₄ and concentrated to give compound B7 (130 mg, 0.16 mmol). LCMS: 786 [M+H]⁺.

### Step 5:

Compound B7 (130 mg, 0.16 mmol) was dissolved in 2 ml DCM and TFA (1 ml) was added. The reaction mixture was stirred at room temperature for 1 hour and concentrated to give final product GTFA-2 (110 mg, 0.15 mmol). C35H63N5O11 LCMS: 730 [M+H]⁺. ¹H NMR (400 MHz, CDCl₃) δ: 4.60-4.35 (m, 2H), 4.21-4.15 (m, 1H), 3.79-3.61 (m, 20H), 3.54-3.45 (m, 1H), 3.41-3.38 (m, 2H), 3.35-3.28 (m, 1H), 3.05-2.93 (m, 1H), 2.80-2.70 (m, 1H), 2.43-2.33 (m, 4H), 1.68-1.60 (m, 4H), 1.22 (m, 24H).

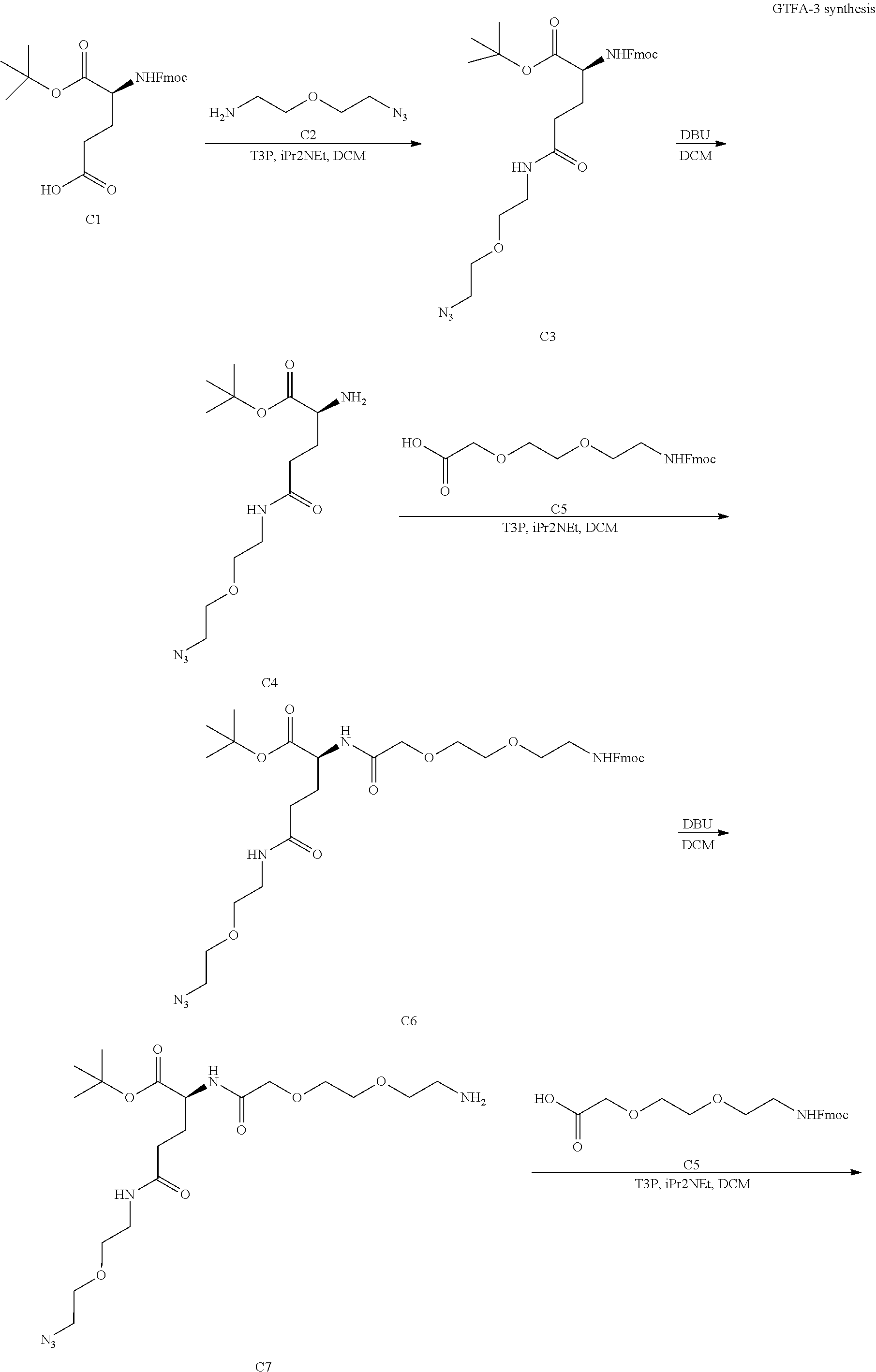

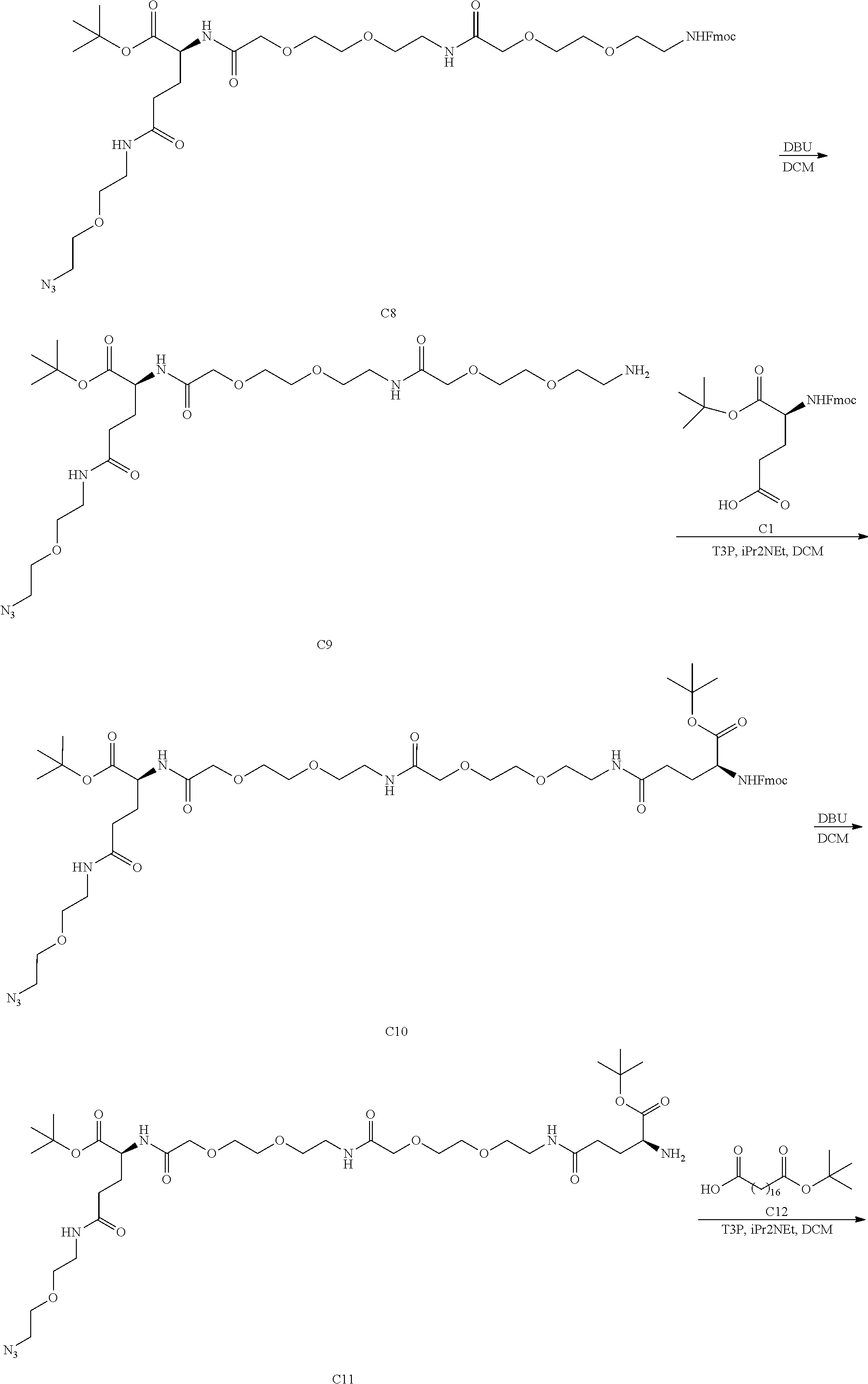

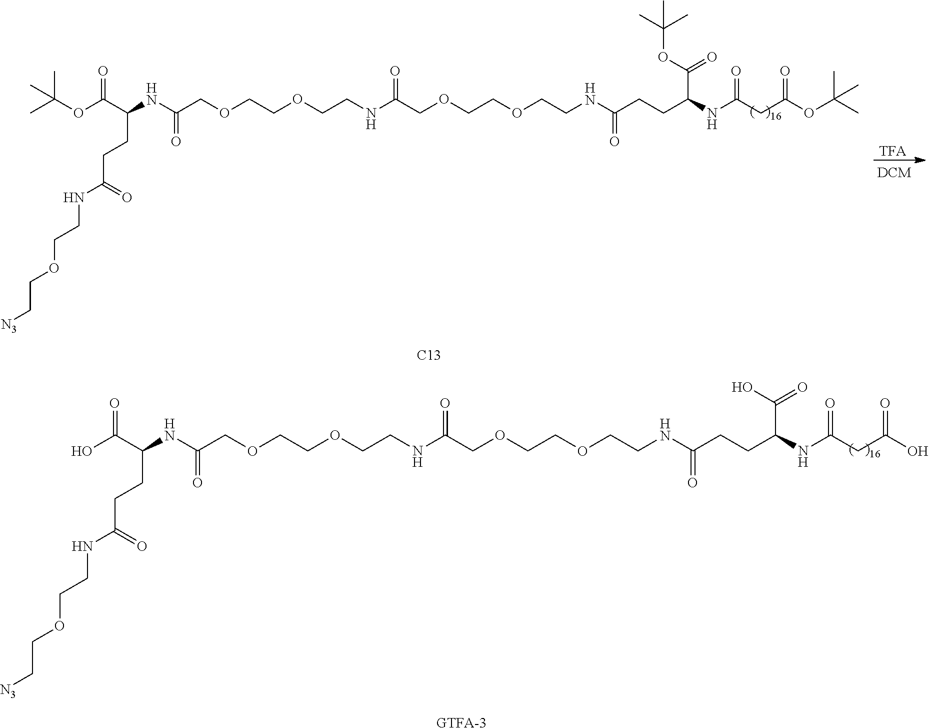

To a solution of compound C1 (3.83 g, 9 mmol) and compound C2 (1.5 g, 9 mmol) in anhydrous THF (50 ml) was added T₃P (7.5 mL, 50% in EA, 1.3 equiv) and DIPEA (5 mL, 3 equiv) at 0° C. The reaction mixture was stirred for 30 minutes and compound C2 (100 mg) was added. The reaction mixture was stirred for 50 minutes and diluted with EtOAc. The mixture was washed with water, brine, dried over Na₂SO₄ and concentrated to get the crude product of compound C3 (˜4.2 g) used for next step. LCMS: 538 [M+H]⁺. Step 2:

To a solution of crude product of compound C3 (˜4.2 g) in DCM (20 mL) at 0° C. was added DBU (3 mL, 19.7 mmol) drop wise. The reaction mixture was stirred for 30 minutes at room temperature and diluted with water. The water layer was extracted with DCM. The combined DCM layer was dried over Na₂SO₄ and concentrated. The residue was added brine and washed with MTBE. The brine layer was extracted with DCM. The combined DCM layer was dried over Na₂SO₄ and concentrated to get the crude product of compound C4 (˜2 g) used for next step. LCMS: 316 [M+H]⁺.

### Step 3:

To a solution of crude product of compound C4 (˜2 g) and compound C5 (1.7 g, 4.4 mmol) in EtOAc (150 ml) was added T₃P (7.5 mL, 50% in EA) and DIPEA (7 mL) at 0° C. The reaction mixture was stirred for 30 minutes and diluted with EtOAc. The mixture was washed with water, brine, dried over Na₂SO₄ and concentrated to get the crude product of compound C6 (˜3.6 g) used for next step. LCMS: 683 [M+H]⁺.

### Step 4:

To a solution of crude product of compound C6 (˜3.6 g) in DCM (6 mL) at 0° C. was added DBU (1 mL, 6.5 mmol) drop wise. The reaction mixture was stirred for 10 minutes at room temperature and diluted with DCM (100 mL) and brine (40 mL). The brine was extracted with DCM and the combined DCM layer was dried over Na₂SO₄ and concentrated. The residue was added water and washed with hexane. The water layer was saturated with NaCl and was extracted with EtOAc. The combined EtOAc layer was dried over Na₂SO₄ and concentrated to get the crude product of compound C7 (˜1.6 g) used for next step. LCMS: 461 [M+H]⁺.

### Step 5:

To a solution of crude product of compound C7 (˜1.6 g) and compound C5 (1.18 g, 3.06 mmol) in EtOAc (200 ml) was added T₃P (4 mL, 50% in EA) and DIPEA (3 mL) at 0° C. The reaction mixture was stirred for 180 minutes and diluted with EtOAc. The mixture was washed with water, brine, dried over Na₂SO₄ and concentrated to get the crude product of compound C8 (˜2.5 g) used for next step. LCMS: 828 [M+H]⁺.

### Step 6:

To a solution of crude product of compound C8 (˜2.5 g) in DCM (3 mL) at 0° C. was added DBU (0.3 mL, 1.98 mmol) drop wise. The reaction mixture was stirred for 2 hours at room temperature and diluted with DCM (100 mL) and brine (40 mL). The brine was extracted with DCM and the combined DCM layer was dried over Na₂SO₄ and concentrated. The residue was added water and washed with hexane. The water layer was saturated with NaCl and was extracted with EtOAc. The combined EtOAc layer was dried over Na₂SO₄ and concentrated to get the crude product of compound C9 (˜1.5 g) used for next step. LCMS: 606 [M+H]⁺.

### Step 7:

To a solution of crude product of compound C9 (˜1.5 g) and compound C1 (0.89 g, 2.1 mmol) in DCM (60 ml) was added T₃P (3 mL, 50% in EA) and DIPEA (2 mL) at 0° C. The reaction mixture was stirred for 120 minutes and diluted with EtOAc. The mixture was washed with water, brine, dried over Na₂SO₄ and concentrated to get the crude product of compound C10 (˜2.4 g) used for next step. LCMS: 1013 [M+H]⁺.

### Step 8:

To a solution of crude product of compound C10 (˜2.4 g) in DCM (2 mL) at 0° C. was added DBU (0.3 mL, 1.98 mmol) drop wise. The reaction mixture was stirred for 2 hours at room temperature and diluted with DCM (100 mL) and brine (40 mL). The brine was extracted with DCM and the combined DCM layer was dried over Na₂SO₄ and concentrated. The residue was added water and washed with hexane. The water layer was saturated with NaCl and was extracted with EtOAc. The combined EtOAc layer was dried over Na₂SO₄ and concentrated to get the crude product of compound C11 (˜1.49 g) used for next step. LCMS: 780 [M+H]⁺.

### Step 9:

To a solution of crude product of compound C9 (˜1.49 g) and compound C12 (0.584 g, 2.1 mmol) in DCM (30 ml) was added T₃P (2 mL, 50% in EA) and DIPEA (2 mL) at 0° C. The reaction mixture was stirred for 60 minutes and diluted with EtOAc. The mixture was washed with water, brine, dried over Na₂SO₄ and concentrated. The residue was purified by silica gel column chromatography with EtOAc/Methanol (20:1) to get compound C13 (1.58 g) as a colorless oil. LCMS: 572 [M+2H]⁺.

### Step 10:

Compound C13 (807 mg, 0.7 mmol) was dissolved in dioxane (3 mL) and HCl (6 ml, 4 N in dioxane, 2.4 mmol) was added. The reaction mixture was stirred at room temperature for 6 hours and concentrated to give compound GTFA-3 (576 mg). C44H78N8O16 LCMS: 975 [M+H]⁺. ¹H NMR (400 MHz, DMSO-d₆) δ: 13.00-11.00 (br, 3H), 8.01-8.03 (m, 1H), 7.90-7.85 (m, 3H), 7.68-7.65 (m, 1H), 4.25-4.21 (m, 1H), 4.14-4.09 (m, 1H), 3.91 (s, 2H), 3.86 (s, 2H), 3.57-3.52 (m, 10H), 3.46-3.36 (m, 8H), 3.29-3.25 (m, 2H), 3.21-3.16 (m, 4H), 2.18-2.05 (m, 8H), 2.04-1.7 (m, 4H), 1.47-1.45 (m, 4H), 1.22 (s, 24H).

### Example 2: Fatty Acid Conjugation to BT100

The core sequence of BT100 (SEQ ID NO. 1) was conjugated with three different fatty acids to create BT500, BT600 and BT700, respectively.

A DBCO moiety was first incorporated to the amino end of BT100. HPLC-purified BT100 with 5′ amino group (amino modifier C6) was reacted with 2.5 molar equivalents of DBCO-Sulfo-NHS ester (CAS #1400191-52-7, Broadpharm, Cat #BP-22289) at room temperature overnight in 100 mM sodium borate buffer pH8.0.

After purified by anion exchange HPLC and without pH adjustment, DBCO labeled BT100 was reacted with 2.5 molar equivalents of palmitic acid, GTFA1 or GTFA3, at room temperature overnight. BT500 (palmitic acid conjugation to BT100), BT600 (GTFA1 conjugation to BT100), or BT700 (GTFA3 conjugation to BT100) was obtained after anion exchange HPLC purification.

BT500: The DBCO labeled aptamer sequence (BT100) was interacted with azido palmitic acid, a saturated C16 fatty acid with nitrogen at the end and one free carboxyl group (—COOH) at the other end. The DBCO moiety reacts with an azide to produce a stable triazole, forming a C16 fatty acid conjugated aptamer with one free COOH group at one end (BT500; SEQ ID NO.: 3)

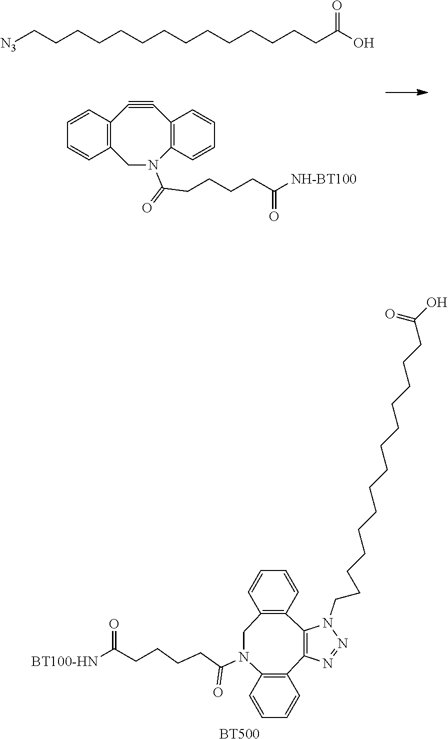

BT600: The DBCO labeled BT100 was attached with octadecanoic diacid through a short polyethylene glycol (PEG) spacer and a γ-glutamic acid linker (GTFA-1), forming a conjugate containing a bi-carboxyl fatty acid (BT600; SEQ ID NO.: 4). The resulted fatty acid-BT100 aptamer compound contains two free COOH groups.

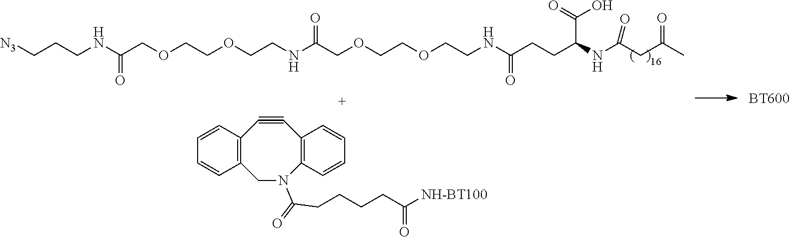

BT700: The DBCO labeled BT100 is conjugated with a tri-carboxyl fatty acid (GTFA-3 which contains three free COOH) (Chemical formula: C44H78N8O16), forming BT700 (SEQ ID NO.: 5).

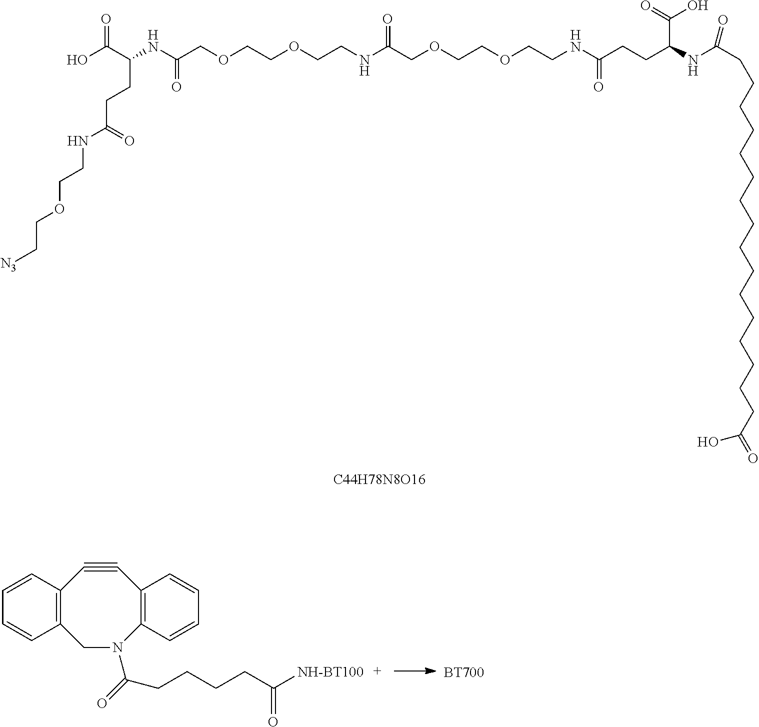

TABLE 1
Sequences of BT500, BT600 and BT700

| | | SEQ |
|---|---|---|
| | Sequence (5′-3′) | ID NO |
| BT100 | (5′) NH2-mGmCmCmAmGmGmGm | 1 |
| | AmCmCmUmAmAmGmAmCmAmCmAm | |
| | UmGmUmCmCmCmUmGmGmC-idT | |
| BT200 | (5′-) PEG40K-mGmCmCmAmGmG | 2 |
| | mGmAmCmCmUmAmAmGmAmCmAmC | |
| | mAmUmGmUmCmCmCmUmGmGmC- | |
| | idT (-3′) | |
| BT500 | (5′-)Palmitic acid-linker- | 3 |
| | mGmCmCmAmGmGmGmAmCmCmUmAm | |
| | AmGmAmCmAmCmAmUmGmUmCmCmC | |
| | mUmGmGmC-idT (-3′) | |
| BT600 | (5′-)GTFA1-linker-mGmCmCm | 4 |
| | AmGmGmGmAmCmCmUmAmAmGmAmC | |
| | mAmCmAmUmGmUmCmCmCmUmGmGm | |
| | C-idT (-3′) | |
| BT700 | (5′-)GTFA3-linker-mGmCm | 5 |
| | CmAmGmGmGmAmCmCmUmAmAmGm | |
| | AmCmAmCmAmUmGmUmCmCmCmUm | |
| | GmGmC-idT (-3′) | |

### Example 3: PK-PD Exposure of Fatty Acid Conjugates in Cynomolgus Monkey

To define the impact of fatty acid conjugation on aptamer distribution (half-life) and efficacy in vivo, the pharmacokinetics and pharmacodynamics (PK-PD exposure) parameters are tested.

All the pharmacokinetic (PK) studies were done in cynomolgus monkey. In addition to PK readout, we also have PFA100 (PD) readout that correlated well with PK readout which suggested that fatty acid conjugation did not change BT100 activity. BT200, a 40 k pegylated BT100 (SEQ ID NO.: 2) was used for comparison.

Animal experiments were performed in accordance with animal care ethics approval and guidelines.

BT500 was administered to animals at 1 mg/kg via intravenous injection (iv) (n=3), or alternatively at 1 mg/kg via subcutaneous injection (sc) (n=3). The REAADS vWF activity and vWF antigen level were measured before dosing and at 48 h, 72 h, 96 h and 168 h after administration (Tables 2 and 3). The platelet function analyzer 100 (PFA100) assessed primary hemostasis under shear stress of the collected blood samples collected at those timepoints as PD results for the functionality of BT500 (Table 4). The readout indicates that BT500 can block vWFA1 binding to Gp1B and inhibit the platelet function, thus PFA100 readout increased from the normal of <100 seconds to the max of ˜300 seconds. The readouts are comparable to those observed in BT100 and BT200 administrations.

TABLE 2
BT500 PK parameters (1 mg/kg; iv)

| | Tₘₐₓ | Cₘₐₓ | AUL₀₋₁ | MRT₀₋₁ | Vd | CL |
|---|---|---|---|---|---|---|
| Animal ID | (h) | (ng/ml) | (h*ng/mL) | (h) | (mL/kg) | (mL/h/kg) |
| 33725 | 0.50 | 12000 | 26300 | 1.7 | 0.0 | 0.0 |
| 33742 | 0.50 | 9800 | 21500 | 1.8 | 0.0 | 0.0 |
| 33748 | 0.50 | 10300 | 17300 | 1.5 | 0.0 | 0.0 |
| Mean | 0.5 | 10700 | 21700 | 1.7 | 0.00 | 0.00 |
| SD | 0.0 | 1150 | 4490 | 0.18 | 0.00 | 0.00 |
| N | 3 | 3 | 3 | 3 | 3 | 3 |

TABLE 3
BT500 PK parameters (1 mg/kg; sc)

| | Tₘₐₓ | Cₘₐₓ | AUL₀₋₁ | MRT₀₋₁ | Vd | CL |
|---|---|---|---|---|---|---|
| Animal ID | (h) | (ng/ml) | (h*ng/ml) | (h) | (mL/kg) | (mL/h/kg) |
| 11322 | 2.0 | 3920 | 28600 | 3.2 | 0.0 | 0.0 |
| 11930 | 2.0 | 5420 | 26700 | 2.9 | 0.0 | 0.0 |
| 33816 | 1.0 | 3580 | 30100 | 3.4 | 0.0 | 0.0 |
| Mean | 1.7 | 4310 | 28400 | 3.2 | 0.00 | 0.00 |
| SD | 0.58 | 979 | 1700 | 0.21 | 0.00 | 0.00 |
| N | 3 | 3 | 3 | 3 | 3 | 3 |

TABLE 4
BT500 PFA100 (seconds)

| | PD results | | | | | |
|---|---|---|---|---|---|---|
| Animal ID | Pre | 0.5 h | 1 h | 2 h | 4 h | 8 h |
| 33725 | 62 | 300 | 293 | 290 | 288 | 99 |
| (1 mg/kg BT500; iv) | | | | | | |
| 33742 | 67 | 225 | 235 | 253 | 236 | 119 |
| (1 mg/kg BT500; iv) | | | | | | |
| 33748 | 54 | 300 | 300 | 267 | 300 | 79 |
| (1 mg/kg BT500; iv) | | | | | | |
| 11322 | 64 | 300 | 300 | 300 | 300 | 300 |
| (1 mg/kg BT500; sc) | | | | | | |
| 11930 | 60 | 246 | 254 | 248 | 230 | 216 |
| (1 mg/kg BT500; sc) | | | | | | |
| 33816 | 57 | 282 | 275 | 275 | 260 | 161 |
| (1 mg/kg BT500; sc) | | | | | | |

BT600 was administered to animals at 1 mg/kg via intravenous injection (iv) (n=2), or alternatively at 3 mg/kg via subcutaneous injection (sc) (n=2). The REAADS vWF activity and vWF antigen level were measured before dosing and at 48 h, 72 h, 96 h and 168 h after administration (Tables 5 and 6).

TABLE 5
BT600 PK parameters (1 mg/kg; iv)

| | | Cₘₐₓ | AUL₀₋₁ | | | Vd | CL |
|---|---|---|---|---|---|---|---|
| Animal | Tₘₐₓ | (mg/ | (h*mg/ | T_{1/2} | MRT₀₋₁ | (L/ | (L/ |
| ID | (h) | L) | L) | (h) | (h) | kg) | h/kg) |
| 33742 | 0.50 | 10.5 | 111 | 10 | 7.8 | 0.128 | 0.00861 |
| 33748 | 0.50 | 12.4 | 121 | 8.4 | 6.9 | 0.100 | 0.00827 |
| Mean | 0.5 | 11.5 | 116 | 9.4 | 7.4 | 0.114 | 0.00844 |
| SD | 0.0 | 1.34 | 6.93 | 1.3 | 0.64 | 0.0197 | 0.000243 |
| N | 2 | 2 | 2 | 2 | 2 | 2 | 2 |

TABLE 6
BT600 PK parameters (3 mg/kg; sc)

| | | Cₘₐₓ | AUL₀₋₁ | | | Vd | CL |
|---|---|---|---|---|---|---|---|
| Animal | Tₘₐₓ | (mg/ | (h*mg/ | T_{1/2} | MRT₀₋₁ | (L/ | (L/ |
| ID | (h) | L) | L) | (h) | (h) | kg) | h/kg) |
| 11749 | 6.0 | 7.50 | 295 | 30 | 20 | 0.337 | 0.00777 |
| 11930 | 6.0 | 11.3 | 353 | 18 | 22 | 0.217 | 0.00816 |
| Mean | 6.0 | 9.40 | 324 | 24 | 21 | 0.277 | 0.00797 |
| SD | 0.0 | 2.69 | 40.8 | 8.2 | 0.95 | 0.0844 | 0.000277 |
| N | 2 | 2 | 2 | 2 | 2 | 2 | 2 |

BT600 can last at least 24 hours by either intravenous injection or subcutaneous injection (Table 7).

TABLE 7
BT600 concentrations (μg/L)

| | Time point (h) | | | | | | | | |
|---|---|---|---|---|---|---|---|---|---|
| Animal ID | 0.0 | 0.5 | 1.0 | 2.0 | 6.0 | 24 | 48 | 72 | 96 |
| 33742 | 0.00 | 10.5 | 8.40 | 6.60 | 4.10 | 1.80 | 0.00 | 0.00 | 0.00 |
| (1 mg/kg BT600; iv) | | | | | | | | | |
| 33748 | 0.00 | 12.4 | 10.8 | 8.90 | 4.60 | 1.60 | 0.00 | 0.00 | 0.00 |
| (1 mg/kg BT600; iv) | | | | | | | | | |
| 11749 | 0.00 | 1.40 | 3.00 | 4.80 | 7.50 | 6.10 | 2.90 | 0.00 | 0.00 |
| (3 mg/kg BT600; sc) | | | | | | | | | |
| 11930 | 0.00 | 1.30 | 3.40 | 6.50 | 11.3 | 6.30 | 2.20 | 1.00 | 0.00 |
| (3 mg/kg BT600; sc) | | | | | | | | | |

As compared to BT100 dosing (Table 8), the half-life of BT600 is extended.

TABLE 8
BT100 concentrations (μg/L)

| | Time point (h) | | | | | | |
|---|---|---|---|---|---|---|---|
| Animal ID | 0.0 | 0.083 | 0.5 | 1.0 | 2.0 | 6.0 | 24 |
| 33725 | 0.00 | 79.2 | 12.5 | 5.20 | 3.00 | 0.00 | 0.00 |
| (3 mg/kg BT100; iv) | | | | | | | |
| 33742 | 0.00 | 54.9 | 10.0 | 6.00 | 2.80 | 0.00 | 0.00 |
| (3 mg/kg BT100; iv) | | | | | | | |
| 33748 | 0.00 | 55.0 | 7.80 | 4.00 | 2.20 | 0.00 | 0.00 |
| (3 mg/kg BT100; iv) | | | | | | | |

BT600 administration (e.g., by sc) does not affect FVIII level nor vWF antigen level in the blood, as opposite to the increased FVIII and vWF antigen level in both monkey and human dosed with BT200, a 40 k PEG conjugated BT100 aptamer (Zhu et al., The development and characterization of a long acting anti-thrombotic von Willebrand factor (VWF) aptamer, Thromb Haemost., 2020, 18(5): 1113-1123; https://isth2021.abstractserver.com/program/#/details/presentations/2661; and https://isth2021.abstractserver.com/program/#/details/presentations/1327) (Table 9).

TABLE 9
vWF antigen and FVIII levels in the blood

| Animal | vWF antigen | | | | FVIII | | | |
|---|---|---|---|---|---|---|---|---|
| ID | 0 h | 24 h | 72 h | 168 h | 0 h | 24 h | 72 h | 168 h |
| 11749 (3 mg/ | 126 | 100 | 76 | 84 | 325.8 | 351.1 | 310.2 | 244.1 |
| kg BT600; sc) | | | | | | | | |
| 11930 (3 mg/ | 110 | 94 | 73 | 99 | 409.2 | 425.5 | 431.1 | 364.7 |
| kg BT600; sc) | | | | | | | | |
| Average | | | | | 367.5 | 388.3 | 370.7 | 304.4 |

BT700 was administered to animals at 1 mg/kg via intravenous injection (iv) (n=2), or alternatively at 2 mg/kg via subcutaneous injection (sc) (n=2). BT700 activity was measured by UV-HPLC before dosing and at 0.5 h, 1 h, 2 h, 4 h, 8 h, 24 h, 48 h, and 72 h after administration (Tables 10 and 11).

TABLE 10
BT700 PK parameters (1 mg/kg; iv)

| | | Cₘₐₓ | AUL₀₋₁ | | | Vd | CL |
|---|---|---|---|---|---|---|---|
| Animal | Tₘₐₓ | (ng/ | (h*ng/ | T_{1/2} | MRT₀₋₁ | (mL/ | (mL/ |
| ID | (h) | ml) | ml) | (h) | (h) | kg) | h/kg) |
| 173768C | 1.0 | 44400 | 473000 | 9.1 | 9.9 | 27.6 | 2.11 |
| 176880C | 1.0 | 29500 | 597000 | 16 | 17 | 35.9 | 1.60 |
| Mean | 1.0 | 37000 | 535000 | 12 | 13 | 31.7 | 1.85 |
| SD | 0.0 | 10500 | 87600 | 4.6 | 5.0 | 5.88 | 0.362 |
| N | 2 | 2 | 2 | 2 | 2 | 2 | 2 |

TABLE 11
BT700 PK parameters (2 mg/kg; sc)

| | | Cₘₐₓ | AUL₀₋₁ | | | Vd | CL |
|---|---|---|---|---|---|---|---|
| | Tₘₐₓ | (ng/ | (h*ng/ | T_{1/2} | MRT₀₋₁ | (L/ | (mL/ |
| Animal ID | (h) | ml) | ml) | (h) | (h) | kg) | h/kg) |
| 176321C | 24 | 13800 | 663000 | 31 | 29 | 102 | 2.29 |
| 176967C | 24 | 22600 | 988000 | 23 | 25 | 59.7 | 1.83 |
| Mean | 24 | 18200 | 826000 | 27 | 27 | 81.0 | 2.06 |
| SD | 0.0 | 6220 | 230000 | 5.9 | 3.3 | 30.1 | 0.324 |
| N | 2 | 2 | 2 | 2 | 2 | 2 | 2 |

The platelet function analyzer 100 (PFA100) assessed primary hemostasis under shear stress of the collected blood samples collected at those timepoints as PD results for the functionality of BT700 (Table 12). The readouts indicate that BT700 can also block vWFA1 binding to Gp1B and inhibit the platelet function, thus PFA100 readout increased from the normal of <100 seconds to the max of ˜300 seconds. The readouts are comparable to those observed in BT100 and BT200 administrations.

TABLE 12
BT700 PFA100 (seconds)

| | PD results | | | | | | | | | |
|---|---|---|---|---|---|---|---|---|---|---|
| Animal ID | Pre | 0.5 h | 1 h | 2 h | 4 h | 8 h | 24 h | 48 h | 72 h | 96 h |
| 173768C | 69 | 294 | 300 | 295 | 294 | 290 | 291 | 63 | 57 | / |
| (1 mg/kg; iv) | | | | | | | | | | |
| 176880C | 56 | 299 | 298 | 286 | 293 | 272 | 300 | 146 | 50 | / |
| (1 mg/kg; iv) | | | | | | | | | | |
| 176321C | / | 111 | 300 | 269 | 266 | 253 | 267 | 268 | 300 | 108 |
| (2 mg/kg; sc) | | | | | | | | | | |
| 176967C | 61 | 300 | 286 | 288 | 300 | 290 | 298 | 294 | 300 | 64 |
| (2 mg/kg; sc) | | | | | | | | | | |

BT100 and BT200 were administered to animals at 3 mg/kg via intravenous injection (iv) for comparison (Tables 13 and 14).

TABLE 13
BT100 and BT200 PK parameters (3mg/kg; iv)

| | BT100 | | | | | | |
|---|---|---|---|---|---|---|---|
| | | Cₘₐₓ | AUL₀₋₁ | | | CL | Vd |
| Animal | Tₘₐₓ | (mg/ | (h*μg/ | T_{1/2} | MRT₀₋₄ | (L/ | (L/ |
| ID | (h) | L) | mL) | (h) | (h) | h/kg) | kg) |
| 33725 | 0.083 | 79.2 | 36.9 | 0.78 | 0.37 | 0.087 | 0.098 |
| 33742 | 0.083 | 54.9 | 29.8 | 0.83 | 0.44 | 0.11 | 0.13 |
| 33748 | 0.083 | 55.0 | 25.8 | 0.86 | 0.38 | 0.12 | 0.15 |
| Mean | 0.083 | 63.0 | 30.9 | 0.82 | 0.40 | 0.11 | 0.13 |
| SD | 0.0 | 14.0 | 5.63 | 0.042 | 0.040 | 0.018 | 0.028 |
| N | 3 | 3 | 3 | 3 | 3 | 3 | 3 |
| | BT200 | | | | | | |
| | | Cₘₐₓ | AUL₀₋₁ | | | CL | |
| | Tₘₐₓ | (μg/ | (h*μg/ | T_{1/2} | MRT₀₋₄ | (mL/ | |
| | (h) | mL) | mL) | (h) | (h) | h/kg) | |
| 33725 | 2.0 | 95.1 | 4430 | 70 | 39 | 0.42 | / |
| 33742 | 2.0 | 77.5 | 4270 | 60 | 38 | 0.47 | / |
| 33748 | 2.0 | 83.9 | 3860 | 55 | 35 | 0.56 | / |
| Mean | 2.0 | 85.5 | 4190295 | 62 | 38 | 0.48 | / |
| SD | 0.0 | 8.91 | | 7.9 | 2.3 | 0.074 | / |
| N | 3 | 3 | 3 | 3 | 3 | 3 | / |

TABLE 14
BT100 and BT200 PFA100 readouts (seconds) (3 mg/kg; iv)

| Animal | PD results | | | | | | | | | | |
|---|---|---|---|---|---|---|---|---|---|---|---|
| ID | Pre | 0.083 h | 0.5 h | 1 h | 2 h | 6 h | 24 h | 48 h | 72 h | 96 h | 168 h |
| | BT100 (3 mg/kg; iv) | | | | | | | | | | |
| 33725 | 71 | 268 | 257 | 272 | 238 | 300 | 69 | | | | |
| 33742 | 69 | 245 | 236 | 241 | 231 | 273 | 78 | | | | |
| 33748 | 64 | 286 | 260 | 258 | 252 | 131 | 64 | | | | |
| | BT200 (3 mg/lg; iv) | | | | | | | | | | |
| 33725 | 68 | / | / | / | 290 | 297 | / | 279 | 282 | 279 | 261 |
| 33742 | 65 | / | / | / | 294 | 291 | / | 300 | 256 | 251 | 255 |
| 33748 | 54 | / | / | / | 300 | 300 | / | N/A | 285 | 277 | 284 |

### Example 4: BT600 Oral Bioavailability

Cynomolgus monkeys were directly fed with BT600 conjugate via oral administration (po) at 5 mg/kg dose. BT600 was co-formulated with the absorption enhancer sodium N-(8-[2-hydroxybenzoyl] amino) caprylate (SNAC) for oral taking (5 mg/kg BT600+150 mg SNAC). The oral administration of BT600 yielded systematic exposure by bioanalytical assay using fluorescent hybridization HPLC method which can detect as low as 1 ng/mL BT600 in the tested animals (Tables 15 and 16).

TABLE 15
BT600 oral bioavailability (5 mg/kg BT600 + 150 mgSNAC; po)

| | | Cₘₐₓ | AUL₀₋₁ | | | Vd | CL |
|---|---|---|---|---|---|---|---|
| Animal | Tₘₐₓ | (ng/ | (h*ng/ | T_{1/2} | MRT₀₋₁ | (mL/ | (mL/ |
| ID | (h) | mL) | mL) | (h) | (h) | kg) | h/kg) |
| C1706137 | 4.0 | 158 | 2520 | 19 | 8.9 | 30000 | 1070 |
| C1706138 | 4.0 | 222 | 3470 | 14 | 8.0 | 19400 | 976 |
| Mean | 4.0 | 190 | 2990 | 17 | 8.4 | 24700 | 1020 |
| SD | 0.0 | 45.3 | 668 | 4.0 | 0.58 | 7530 | 68.6 |
| N | 2 | 2 | 2 | 2 | 2 | 2 | 2 |

TABLE 16
BT600 PFA100 (seconds) (5 mg/kg BT600 + 150 mgSNAC)

| | PD results | | | | | | |
|---|---|---|---|---|---|---|---|
| Animal ID | Pre | 0.5 h | 1 h | 2 h | 4 h | 8 h | 24 h |
| C1706137 (male) | 68 | 59 | 72 | 64 | 76 | 58 | 69 |
| C1706138 (female) | * | 55 | 59 | 57 | 58 | 58 | * |

A low bioavailability (0.2%) is detected and the plasma concentration of BT600 is too low to have an impact on PFA100 assay. However, a long half-life of BT600 is observed in the tested animals though the tissue distribution is low.

### Example 5: Fatty Acid Conjugates Binding to Albumin

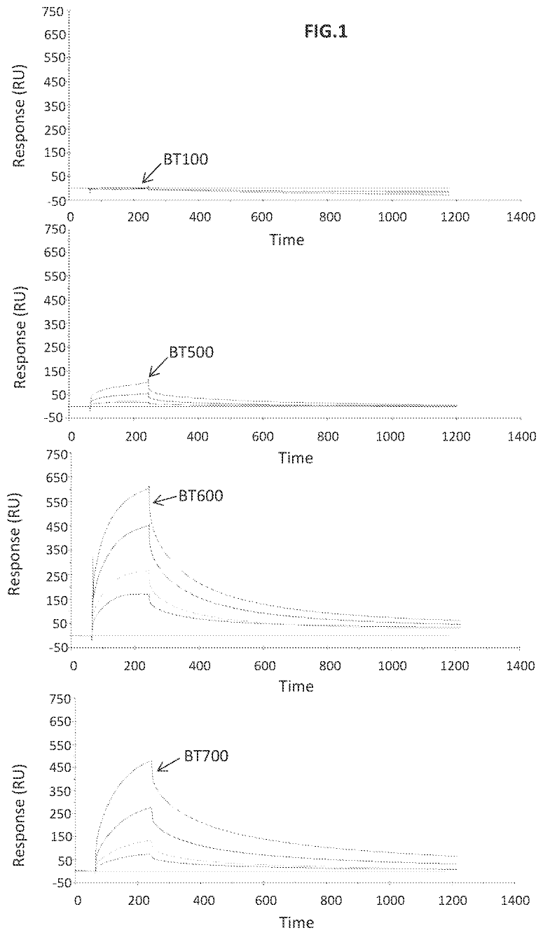

CM5 chips coated with albumin via amine coupling were used. The sensor chip was coated with FC-2 human albumin, FC-3 mouse albumin and FC-4 bovine albumin. FC-1 was used as control. Aptamers and fatty acid conjugates BT500, BT600 and BT700 were run over the surface of the chip. BT100 was used as a control (as shown in FIG. 1).

Based on this simple 1:1 Langmuir interaction model, association constant (ka), dissociation constant (kd) and equilibrium dissociation constant (KD) were calculated and compared. No significant binding is observed, and curved fitting was not applied for BT100, while all three fatty acid conjugates BT500, BT600 and BT700 showed binding to human albumin (Table 17). BT100 does not bind to albumin, thus with the shortest half-life in Monkey. Although BT500 has low affinity toward albumin (FIG. 1), the low binding of albumin still gives BT500 a longer half-life as compared to BT100. BT600 and BT700 have high affinity toward albumin and thus much longer half-life (FIG. 1).

TABLE 17
Human albumin binding of BT compounds

| | ka | kd | KD |
|---|---|---|---|
| Compound ID | (1/Ms) | (1/s) | (M) |
| BT100 | N/A* | N/A* | N/A* |
| BT500 | 2.43 × 10³ | 3.03 × 10⁻³ | 1.25 × 10⁻⁶ |
| BT600 | 8.60 × 10³ | 1.24 × 10⁻³ | 1.44 × 10⁻⁷ |
| BT700 | 3.61 × 10³ | 1.42 × 10⁻³ | 3.93 × 10⁻⁷ |

1. A conjugate comprising a nucleic acid moiety and at least one fatty acid moiety, wherein the fatty acid moiety is conjugated to one end of the nucleic acid moiety, and wherein the fatty acid moiety comprises —(CH₂)ₐ—COOH, wherein a is an integer between 12 and 26.

2. The conjugate of claim 1 comprising at least 1 additional carboxyl group.

3. The conjugate of claim 1 comprising 1 or 2 additional carboxyl groups.

4. The conjugate of claim 1, wherein the fatty acid moiety comprises at least one ethylene glycol group (—(OCH₂CH₂)—).

5. The conjugate of claim 4, wherein the fatty acid moiety comprises between 1 and 10 ethylene glycol groups.

6. The conjugate of claim 1, wherein the fatty acid moiety comprises at least one polyethylene glycol (PEG) group.

7. The conjugate of claim 1, claim 4 or claim 6, wherein the fatty acid moiety comprises at least one amide group (—NH—CO—).

8. The conjugate of claim 7, wherein the fatty acid moiety comprises at least one —(OCH₂CH₂—OCH₂CH₂—NH—CO)— group.

9. The conjugate of claim 7, wherein the fatty acid moiety comprises at least one —(CO—CH₂—OCH₂CH₂—OCH₂CH₂—NH)— group.

10. The conjugate of claim 1, claim 4, claim 6 or claim 7, wherein the fatty acid moiety comprises at least one glutamic acid group.

11. The conjugate of claim 10, wherein the fatty acid moiety comprises

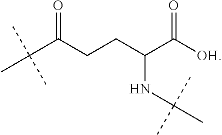

12. The conjugate of claim 10, wherein the glutamic acid group is a γ-glutamic acid group.

13. The conjugate of claim 10, wherein the fatty acid moiety comprises

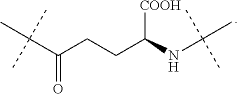

14. The conjugate of claim 1, claim 4, claim 6, claim 7 or claim 10, wherein the fatty acid moiety comprises a piperazine group.

15. The conjugate of claim 14, wherein the fatty acid moiety comprises piperazine-2-carboxylic acid group

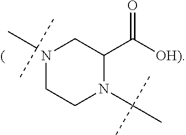

16. The conjugate of claim 14, wherein the fatty acid moiety comprises a 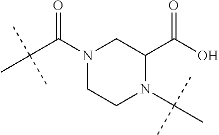
group.

17. The conjugate of claim 14, wherein the fatty acid moiety comprises a 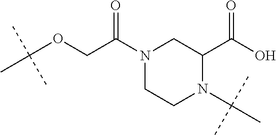
group.

18. The conjugate of claim 1, wherein the fatty acid moiety comprises a structure of: 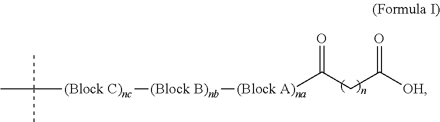
wherein n is an integer from 12 to 26,
Block A is 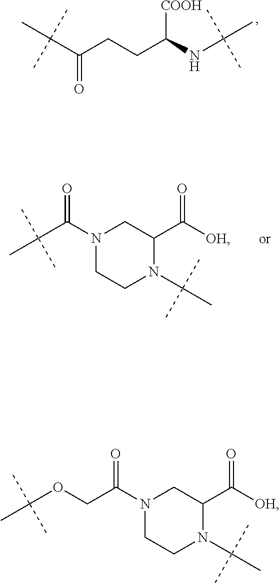
na is 0 or 1,
Block B is —(OCH₂CH₂)— or —(CO—CH₂—OCH₂CH₂—OCH₂CH₂—NH)—,
nb is an integer from 1 to 10,
Block C is 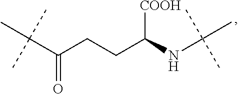
and
nc is 0 or 1.

19. The conjugate of claim 1, wherein the fatty acid moiety comprises GTFA-1′, GTFA-2′, or GTFA-3′.

20. The conjugate of any one of the preceding claims, wherein the nucleic acid molecule is a therapeutic nucleic acid, including an aptamer or a variant thereof, an oligonucleotide, an antisense oligonucleotide, a CpG oligonucleotide, a siRNA, a shRNA, a microRNA, a lncRNA, a mRNA, an antisense RNA, a saRNA, a circular RNA and the like.

21. The conjugate of claim 20, wherein the therapeutic nucleic acid is an aptamer or a variant thereof.

22. The conjugate of claim 21, wherein the aptamer comprises at least one modification; the modifications may be a nucleoside modification or a backbone modification.

23. The conjugate of claim 22, wherein the aptamer comprises at least one nucleoside modification; the modification is 2′-O-methyl modification.

24. A composition comprising a therapeutically effective amount of a conjugate according to any one of claims 1-23, and one or more therapeutically acceptable carriers.

25. A composition comprising an aptamer comprising a nucleic acid sequence and at least one fatty acid moiety, wherein the 5′ end of the nucleic acid sequence of the aptamer has a DBCO group.

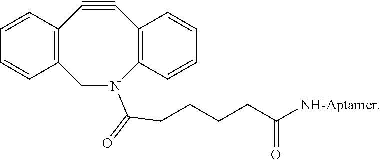

26. The composition of claim 25, wherein the fatty acid moiety is covalently attached to the 5′ end of the nucleic acid sequence of the aptamer.

27. The composition of claim 26, wherein the fatty acid moiety comprises GTFA-1, GTFA-2, or GTFA-3.

28. The composition of any one of claims 25-27, wherein the aptamer comprises a modified nucleoside.

29. The composition of claim 28, wherein the aptamer comprises at least one 2′-O-methyl modified nucleoside.

30. A fatty acid moiety comprises a general formula of: 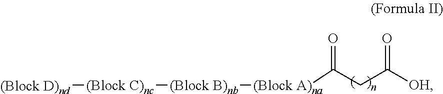
wherein n is an integer from 12 to 26,
Block A is 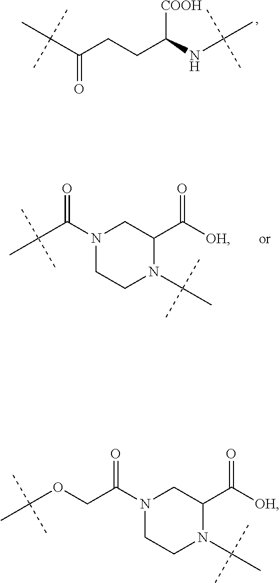
na is 0 or 1,
Block B is —(OCH₂CH₂)— or —(CO—CH₂—OCH₂CH₂—OCH₂CH₂—NH)—,
nb is an integer from 1 to 10,
Block C is 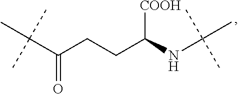
nc is 0 or 1,
Block D comprise a functional group that can covalently bind to a nucleic acid, and
nd is 0 or 1.

31. The fatty acid moiety of claim 30, wherein Block D comprises an azide group, an alkyne group, a hydroxy group, a sulfhydryl group, or an amino group.

32. The fatty acid moiety of claim 30, wherein Block D is R_{d}—N₃, wherein R_{d} comprises an alkyl group, an amino alkyl group, an amine group, and/or an alkoxyl group.

33. The fatty acid moiety of claim 32, wherein Block D is

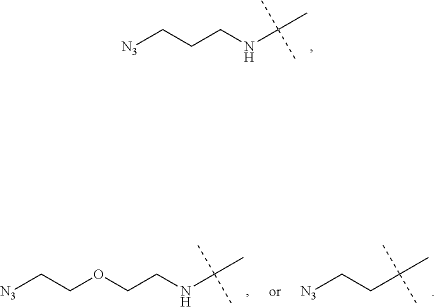

34. The fatty acid moiety of claim 30, wherein the fatty acid moiety is GTFA-1, GTFA-2, or GTFA-3.

35. A polynucleotide comprising a nucleic acid sequence presented by SEQ ID NO: 1 and a fatty acid moiety conjugated to one terminal of the sequence of SEQ ID NO:1, wherein the fatty acid moiety is selected from the group consisting of octadecanoic diacid, GTFA-1, GTFA-2 and GTFA-3.

36. The polynucleotide of claim 35 comprising the nucleic acid sequence presented by SEQ ID NO: 3 and Palmitic Acid that is conjugated to the 5′ terminal of SEQ ID NO: 3.

37. The polynucleotide of claim 35 comprising the nucleic acid sequence presented by SEQ ID NO: 4 and GTFA-1 that is conjugated to the 5′ terminal of SEQ ID NO: 4.

38. The polynucleotide of claim 35 comprising the nucleic acid sequence presented by SEQ ID NO: 5 and GTFA-3 that is conjugated to the 5′ terminal of SEQ ID NO: 5.

39. A pharmaceutical composition comprising the polynucleotide of any one of claims 35-38, and a pharmaceutical acceptable carrier.
